(19) United States
(12) Patent Application Publication
(10) Pub. No.: US 2004/0132789 A1
(43) Pub. Date: Jul. 8, 2004

(54) Antioxidant and radical scavenging activity of synthetic analogs of desferrithiocin

(75) Inventor: Raymond J. Bergeron JR., Gainesville, FL (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: University of Florida, Gainesville, FL

(21) Appl. No.: 10/645,348

(22) Filed: Aug. 21, 2003

Related U.S. Application Data
(63) Continuation-in-part of application No. 10/227,158, filed on Aug. 22, 2002.
(60) Provisional application No. 60/405,463, filed on Aug. 22, 2002.

(51) Int. Cl. A61K 31/4439; A61K 31/426

(52) U.S. Cl. 514/367; 514/369; 514/342

(57) ABSTRACT

Free radicals and reactive oxygen species have the potential to damage a wide variety of organic molecules, typically by oxidizing certain moieties. The present invention includes methods of using aryl-substituted heterocyclic compounds as antioxidants, as well as suppressing the formation of radical species. In addition, the present invention provides methods of treating conditions such as inflammatory disease, neoplastic disease, and ischemic episodes.

### RELATED APPLICATIONS

This application is a continuation-in-part of U.S. application Ser. No. 10/227,158, filed Aug. 22, 2002. This application also claims the benefit of U.S. Provisional Application No. 60/405,463, filed on Aug. 22, 2002. The entire teachings of the above applications are incorporated herein by reference.

### GOVERNMENT SUPPORT

The invention was supported, in whole or in part, by grant R01-DK49108 from the National Institutes of Health. The Government has certain rights in the invention.

### BACKGROUND OF THE INVENTION

Free radicals and reactive oxygen species (ROS) are normal by-products of cellular respiration. For example, it has been estimated that 90% of the oxygen used by activated neutrophils is converted to superoxide anion by NADPH oxidase, and that the concentration of this free radical and other ROS can reach concentrations as high as 1.25 M at the neutrophil substrate cleft. While some of these free radicals and ROS can serve as signaling molecules or in other regulatory functions at normal physiological concentrations, elevated levels of free radicals and/or ROS are typically toxic. Toxicity from superoxide anion can result from dismutation to water and hydrogen peroxide followed by reaction of hydrogen peroxide with myeloperoxidase and chloride to produce hypochlorous acid (HOCI), a highly toxic substance.

An organism typically has both enzymatic (e.g., superoxide dismutase, catalase) and non-enzymatic (e.g., ascorbate, glutathione) defenses against elevated free radical and ROS levels. Nevertheless, under some circumstances, the defenses against free radicals and/or ROS are depleted or overwhelmed, which initiates or contributes to cellular damage. Types of cellular damage include DNA strand breaks, DNA base cleavage, protein oxidation, and lipid membrane oxidation. Outside of an organism, free radicals and ROS contribute to the degradation or spoilage of organic compounds, typically by oxidation or peroxidation of a compound.

One pathway through which free radicals and ROS form is when reduced iron and hydrogen peroxide react. This reaction is known as the Fenton reaction, and it produces a hydroxyl radical, a species that reacts at a diffusion-controlled rate with most organic compounds. One way of preventing the Fenton reaction is by stabilizing the iron(III) electronic state. Therefore, the stabilization of iron(III) may be beneficial in decreasing damage or degradation due to the Fenton reaction.

Other pathways leading to free radical formation, such as the enzymes NADPH oxidase, xanthine oxidase, NADH oxidase, aldehyde oxidase, and dihydroorotate dehydrogenase, are difficult or impossible to inhibit without deleterious effects on an organism. Therefore, it is desirable to develop antioxidant compounds that can directly quench (e.g., reduce or oxidize) certain radical species, depending on the reduction potential of the radical. By directly quenching a free radical, the antioxidant compounds will prevent damage to cells or organic molecules.

There is a need for compounds that suppress the formation of free radicals and quench free radicals. Ideally, there is a class of compounds that has these functions.

### SUMMARY OF THE INVENTION

It has now been found that a variety of aryl-substituted heterocycles, the structures of which are shown below, are able to inhibit the reduction of iron(I1I) to iron(II) in the presence of ascorbate (Example 1). It has additionally been found that such compounds can quench a radical species, 2,2′-azinobis(3-ethylbenzothiazoline-6-sulfonic acid) (ABTS⁺) (Example 2). Compounds of the present invention have the potential to limit the formation of and damage caused by free radicals and ROS by multiple mechanisms.

The present invention includes a method of suppressing radical formation in vitro, comprising the step of contacting a solution with a compound represented by Structural Formula (I):

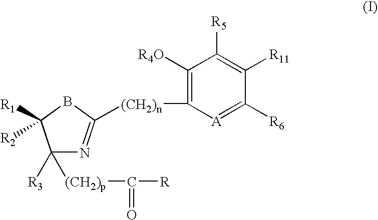

where:

R is —OH, —OR₇, —N(OH)R₈;

R₁ is —H, —CH₃, or an alkyl of 1-6 carbons;

R₂ is —H, —CH₃, or an alkyl of 1-6 carbons;

R₃ is —H, —CH₃, an alkyl of 1-6 carbons, or R₂ and R₃ together form a double bond;

R₄ is —H, acyl of 1-4 carbons, or alkyl of 1-4 carbons;

R₅ is —H, —OH, —O-acyl of 1-4 carbons, —O-alkyl of 1-4 carbons, or —L—X;

R₆ is —H, —OH, alkyl of 1-6 carbons, a halogen, —L—Y, or Rr is —C═C—C═C—, which together with R₁₁ forms a fused ring system as follows:

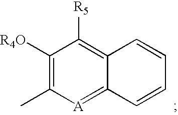

R₇ is alkyl of one to four carbons or optionally substituted benzyl;

R₈ is —H, alkyl of one to four carbons, optionally substituted benzyl,

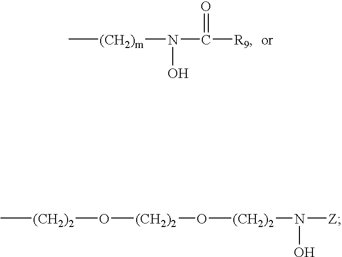

preferably, R₈ is —H, alkyl of one to four carbons, optionally substituted benzyl, or

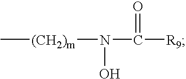

R₉ is —H, alkyl of one to four carbons, or optionally substituted benzyl;

R₁₁ is —H, —OH, —O-acyl of 1-4 carbons, or —O-alkyl of 1-4 carbons;

A is N, CH, or C(OH);

B is S, O, NR₉, CH₂ or CH₂S;

L is an alkylene group of 3 to about 20 carbon atoms which is optionally interrupted by one or more oxygen atoms;

a is 2 or 3;

m is an integer from 1 to 8;

n is 0, 1 or 2;

p is 0, 1 or 2;

X is

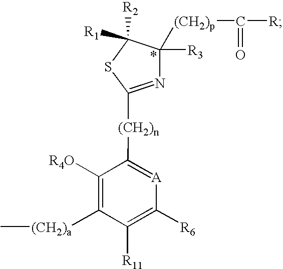

Y is

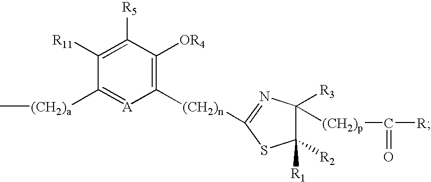

Z is

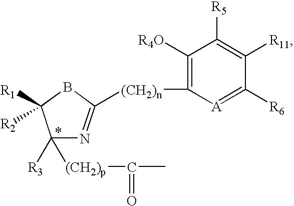

where each of the substituents shown is defined above; or a compound of formula (I) wherein the ring containing the B and N moieties is fully reduced and contains no double bonds; or a pharmaceutically acceptable salt of the compound represented by formula (I) or a stereoisomer of the compound or mixture of stereoisomers; with the proviso that when R is —OH, R₁ and R₂ are —H, R₃ is —CH₃, R₄, R₅, R₆, and R₁ ₁ are —H, A is N, and B is S, then n and p are not 0.

In another embodiment, the present invention is a method of suppressing radical formation in vitro, comprising the step of contacting a solution with a compound represented by Structural Formula (II) or Structural Formula (III):

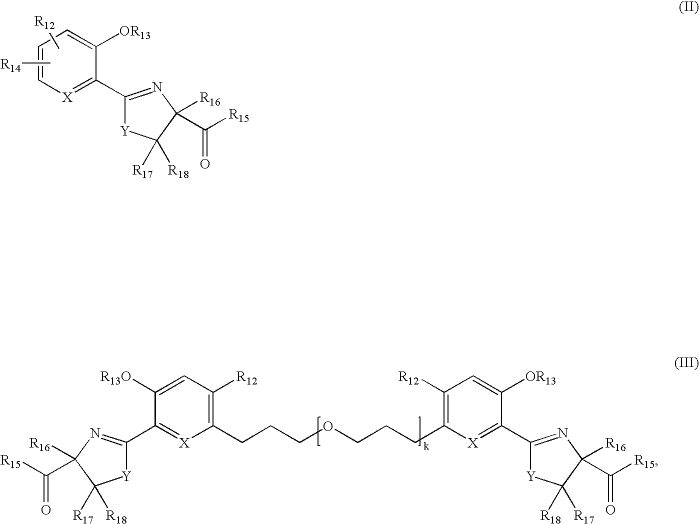

where:

R₁₂ is —H, —OR₁₉, or an —O-acyl group;

R₁₃ is —H, —R₁₉, or an acyl group;

R₁₄, R₁₆, R₁₇, R₁₈, R₁₉, and R₂₀ are each independently —H or a lower substituted or unsubstituted alkyl group, or R₁₆ and R₁₈ together form a double bond;

R₁₅ is —OH, —OR₂₀, or —N(R₂₀)OH;

X is CH or N;

Y is S, CH, O, NR₂₀, or SCH₂; and

k is an integer; or a pharmaceutically acceptable salt thereof, provided that for Structural Formula (II) when R₁₂, R₁₃, and R₁₄ are —H, R₁₅ is —OH, R₁₆ is —H₃, R₁₇ and R₁₈ are —H, and X is N, then Y is not S.

In yet another embodiment of the invention, the compound is represented by Structural Formula (IV):

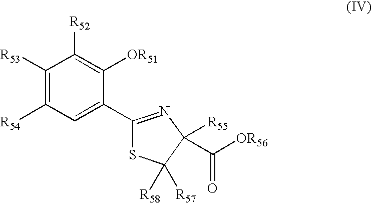

where:

R₅, is —H, alkyl, or alkanoyl;

R₅₂, R₅₃, and R₅₄ are each independently —H, hydroxy, alkoxy, or alkanoyloxy; and

R₅₅, R₅₆, R₅₇, and R₅₈ are each independently —H or alkyl.

In addition, the present invention is a method of treating a patient to suppress formation of radical species; and a method of treating a patient in need of antioxidant therapy, comprising the step of administering to said patient a therapeutically effective amount of a compound represented by Structural Formula (I), (II), (III) or (IV). In yet another embodiment, the present invention is a method of treating a patient who is suffering from neoplastic disease or a preneoplastic condition, comprising the step of administering to said patient a therapeutically effective of a compound represented by Structural Formula (I), (II), (III) or (IV).

The present invention also includes a method of preventing or inhibiting oxidation of a substance in vitro, comprising the step of contacting said substance with an effective amount of a compound represented by Structural Formula (I), (II), (III) or (IV).

In addition, the present invention provides a method of scavenging free radicals, comprising the step of contacting said free radicals with a compound represented by Structural Formula (I), (II), (III) or (IV). Free radicals can be scavenged in vitro or in vivo, for example, to prevent or inhibit free radical-mediated damage to cells, tissues or organs.

In another aspect, the present invention is a method of treating a patient who is suffering from, has suffered from, or is at risk of suffering from an ischemic episode and a method of treating a patient who is suffering from an inflammatory disorder, provided said inflammatory disorder is not inflammatory bowel disorder, comprising the step of administering to said patient a therapeutically effective amount of a compound represented by Structural Formula (I):

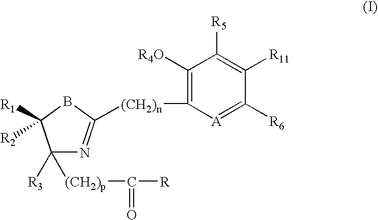

wherein:

R is —OH, —OR₇ or —N(OH)R₈;

R₁ is —H, —CH₃, or an alkyl of 1-6 carbons;

R₂ is —H, —CH₃, or an alkyl of 1-6 carbons;

R₃ is —H, —CH₃, or an alkyl of 1-6 carbons, or R₂ and R₃ together form a double bond;

R₄ is —H, acyl of 1-4 carbons, or alkyl of 1-4 carbons;

R₅ is —H, —OH, —O-acyl of 1-4 carbons, —O-alkyl of 1-4 carbons, or —L—X;

R₆ is —H, —OH, alkyl of 1-6 carbons, a halogen, —L—Y, or R₆ is —C═C—C═C—, which together with R₁₁ forms a fused ring system as follows:

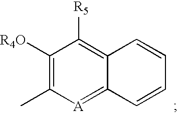

R₇ is alkyl of one to four carbons or optionally substituted benzyl;

R₈ is —H, alkyl of one to four carbons, optionally substituted benzyl,

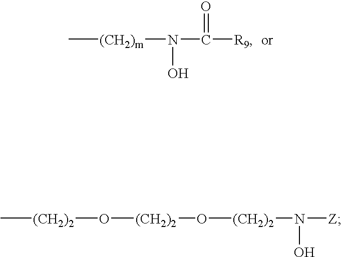

R₉ is —H, alkyl of one to four carbons, or optionally substituted benzyl;

R₁₁ is —H, —OH, —O-acyl of 1-4 carbons, or —O-alkyl of 1-4 carbons;

A is N, CH, or C(OH);

B is S, O, NR₉, CH₂ or CH₂S;

a is 2 or 3;

L is an alkylene group of 3 to about 20 carbon atoms which is optionally interrupted by one or more oxygen atoms;

m is an integer from 1 to 8;

n is 0, 1 or 2;

p is 0, 1 or 2;

X is

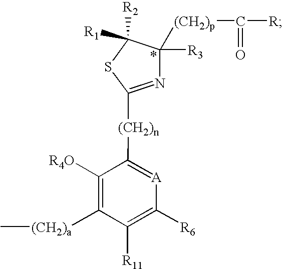

Y is

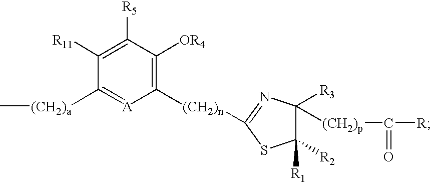

Z is

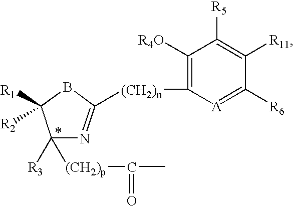

wherein each of the substituents shown is defined above; or a compound of formula (I) wherein the ring containing the B and N moieties is fully reduced and contains no double bonds; or a pharmaceutically acceptable salt of the compound represented by formula (I) or a stereoisomer of the compound or mixture of stereoisomers.

In another embodiment, the present invention includes a method of treating a patient who is suffering from, has suffered from, or is at risk of suffering from an ischemic episode, and a method of treating a patient who is suffering from an inflammatory disorder, provided said inflammatory disorder is not inflammatory bowel disorder, comprising the step of administering to said patient a therapeutically effective amount of a compound represented by Structural Formula (II) or Structural Formula (III):

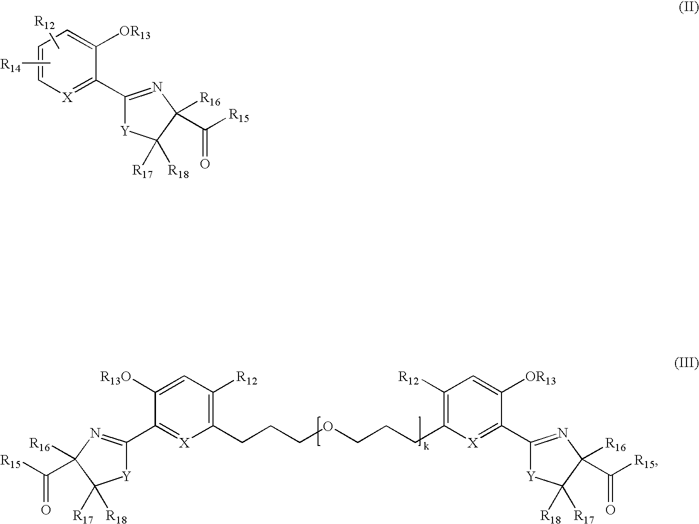

where:

R₁₂ is —H, —OR₁₉, or an —O-acyl group;

R₁₃ is —H, —R₁₉, or an acyl group;

R₁₄, R₁₆, R₁₇, R₁₈, R₁₉, and R₂₀ are each independently —H or a lower substituted or unsubstituted alkyl group, or R₁₆ and R₁₈ together form a double bond;

R₁₅ is —OH, —OR₂₀, or —N(R₂₀)OH;

X is CH or N;

Y is S, CH, O, NR₂₀, or SCH₂; and

k is an integer;

provided that for Structural Formula (II) when R₁₂, R₁₃ and R₁₄ are —H, R₁₅ is —OH, R₁₆ is —CH₃, R₁₇ and R₁₈ are —H, and X is N, then Y is not S.

In a further embodiment, the method of treating a patient who is suffering from, has suffered from or is at risk of suffering from an ischemic episode comprises administering a therapeutically effective amount of a compound represented by Structural Formula (IV).

Advantages of the present invention include providing compounds that can serve as antioxidants by quenching and suppressing formation of free radicals. Compounds of the present invention can be modified at various locations in the molecule in order to improve antioxidant properties.

### BRIEF DESCRIPTION OF THE DRAWINGS

FIGS. 1A and 1B show the effect of various compounds on the iron-mediated oxidation of ascorbate.

FIG. 2 shows representative colons (n=3 from each group) from rats treated with (1) no test compound (water) and 4% acetic acid, (2) desferrioxamine 30 minutes before the 4% acetic acid, and (3) Rowasa® 30 minutes before the 4% acetic acid.

FIG. 3 shows a synthetic scheme for (S,S)-1,11-Bis[5-(4-carboxy-4,5-dihydro-thiazol-2-yl)-2,4-dihydroxyphenyl]-4,8-dioxaundecane.

FIG. 4 shows the effect of various compounds on the iron-mediated oxidation of ascorbate.

FIG. 5 shows the effect of various compounds on the iron-mediated oxidation of ascorbate.

FIG. 6 shows the ABTS radical cation quenching activity of selected desferrithiocin analogs, therapeutic iron chelators, and 5-aminosalicylic acid versus that of Trolox.

FIG. 7 shows the efficacy of compounds in preventing visible and biochemical colonic damage in rats.

FIG. 8 shows the ABTS radical cation quenching activity of selected compounds.

FIG. 9 shows the ABTS radical cation quenching activity of selected compounds.

### DETAILED DESCRIPTION OF THE INVENTION

The present invention relates to compounds that act as antioxidants, either by directly interacting with a radical or oxidant species or by interacting with an atom or molecule (e.g., a redox-active metal ion) capable of generating radical and/or oxidant species. These compounds can be administered to a patient to treat a variety of conditions, including ischemic episodes, inflammatory disease, neoplastic disease, and preneoplastic conditions.

Compounds of the present invention are represented by Structural Formulae (I), (II), and (III), as shown above. R₁₂ is preferably —H, —OH, or —OCH₃ and/or R₁₃ is preferably —H. Preferred examples of R₁₃, R₁₄, R₁₇, R₁₈, and R₂₀ are —H and —CH₃, particularly when R₁₂ and/or R₁₃ have the preferred values indicated above. A preferred R₁₅ is —OH or —N(R₁₈OH), and k is preferably 1 or 2, particularly when R₁₂, R₁₃, R₁₄, R₁₇, R₁₈ and/or R₂₀ have the preferred values shown hereinabove.

Compounds of the present invention are also represented by Structural Formula (IV), as shown above. R₅, is preferably —H, —CH₃, or —C(O)CH₃. Preferred examples of R₅₂, R₅₃, and R₅₄ include —H, —OH, —OCH₃, and —OC(O)CH₃, particularly when R₅₁ has one of the above-listed preferred values. Preferably, R₅₅, R₅₆, R₅₇, and R₅₈ are each independently —H or —CH₃, especially when R₅₁, R₅₂, R₅₃ and/or R₅₄ have the preferred values described above.

Other suitable compounds are represented by Structural Formulae (V) to (XVI), with alternative names indicated as follows:

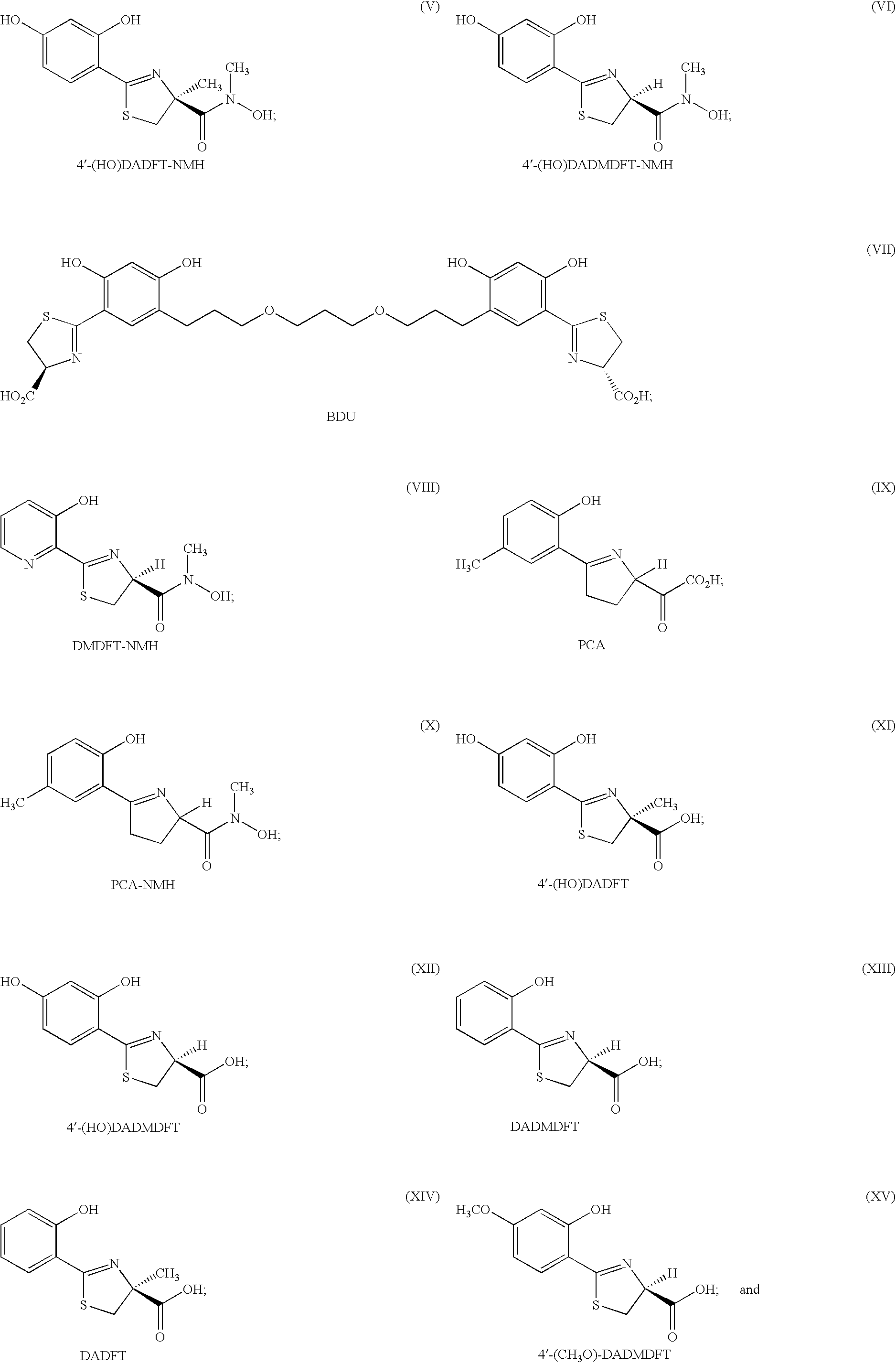

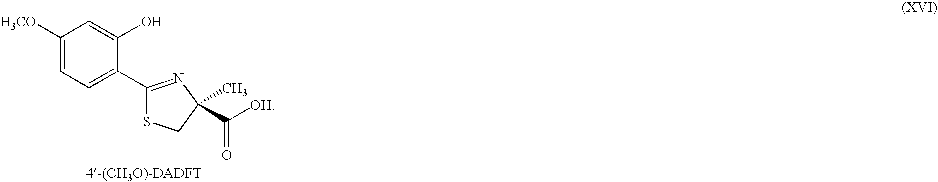

Other compounds for use in the present invention are represented by Structural Formulas (XVII) to (XX):

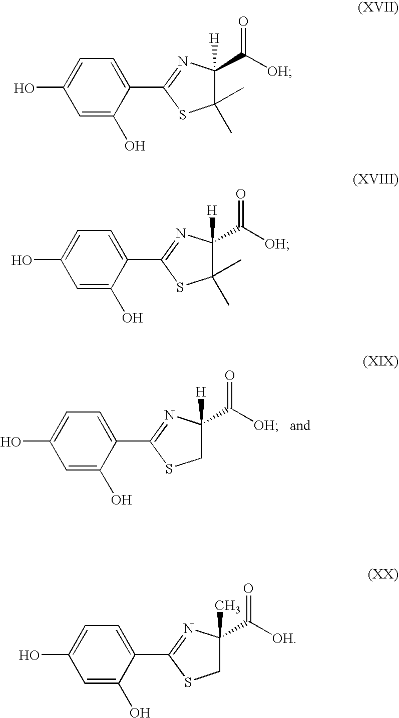

Additional compounds for use in the present invention can be found in pending. applications U.S. Ser. Nos. 09/531,753, filed Mar. 20, 2000 (now U.S. Pat. No. 6,559,315), 09/531,755, filed Mar. 20, 2000 (now U.S. Pat. No. 6,525,080), and 09/723,809 (now abandoned), filed Nov. 28, 2000, as well as U.S. Pat. Nos. 5,840,739, 6,083,966 and 6,521,652, all of which are incorporated herein by reference.

In some embodiments of the invention, the compounds used in the methods described herein optionally do not include desferrithiocin, (R)-desmethyldesferrithiocin, (S)-desazadesmethyldesferrithiocin and/or (S)-desmethyldesferrithiocin.

Stereoisomers of the compounds represented by Structural Formulas (I) to (XX), such as enantiomers and diastereomers, are suitable for use in the present invention. In addition, racemic mixtures of the above compounds are suitable for use in the present invention. In instances where more than one, or more than two stereoisomers of a compound are present, mixtures of the stereoisomers are acceptable.

If desired, mixtures of stereoisomers can be separated to form an optically-active compound (with respect to any optically-active carbon center). In one example, a compound comprising an acid moiety can be resolved by forming a diastereomeric salt with a chiral amine. Suitable chiral amines include arylalkylamines such as (R)-1-phenylethylamine, (S)-1-phenylethylamine, (1R)-1-tolylethylamine, (S)-1-tolylethylamine, (R)-1-phenylpropylamine, (S)-1-propylamine, (R)-1-tolylpropylamine, and (S)-1-tolylpropylamine. Resolution of chiral compounds using diastereomeric salts is further described in CRC Handbook of Optical Resolutions via Diastereomeric Salt Formation by David Kozma (CRC Press, 2001), which is incorporated herein by reference in its entirety.

An alkyl group is a saturated hydrocarbon in a molecule that is bonded to one other group in the molecule through a single covalent bond from one of its carbon atoms. Alkyl groups can be cyclic or acyclic, branched or unbranched, and saturated or unsaturated. Typically, an alkyl group has one to about six carbon atoms, or one to about four carbon atoms. Lower alkyl groups have one to four carbon atoms and include methyl, ethyl, n-propyl, iso-propyl, n-butyl, sec-butyl and tert-butyl.

Acyl or alkanoyl groups are represented by the formula —C(O)R, where R is a substituted or unsubstituted alkyl group. Acyloxy or alkanoyloxy groups (i.e., —O-acyl) are represented by the formula —O—C(O)R. Acyl (alkanoyl) or, preferably, acyloxy (alkanoyloxy) groups can be hydrolyzed or cleaved from a compound by an enzyme, acids, or bases. One or more of the hydrogen atoms of an acyl (alkanoyl) or acyloxy (alkanoyloxy) group can be substituted, as described below. Typically, an acyl (alkanoyl) or acyloxy (alkanoyloxy) group is removed before a compound of the present invention binds to a metal ion such as iron(III).

Suitable substituents for alkyl, acyl (alkanoyl), and acyloxy (alkanoyloxy) groups include —OH, —O(R′), —O—CO—(R′), —NO₂, —COOH, ═O, —NH₂, —NH(R′), —N(R′)₂, —COO(R′), —CONH₂, —CONH(R′), —CON(R′)₂, and guanidine. Each R′ is independently an alkyl group or an aryl group. Alkyl groups can additionally be substituted by an aryl group (e.g. an alkyl group can be substituted with an aromatic group to form an arylalkyl group). A substituted alkyl group can have more than one substituent.

Aryl groups include carbocyclic aromatic groups such as phenyl, p-tolyl, 1-naphthyl, 2-naphthyl, 1-anthracyl and 2-anthracyl. Aryl groups also include heteroaromatic groups such as N-imidazolyl, 2-imidazole, 2-thienyl, 3-thienyl, 2-furanyl, 3-furanyl, 2-pyridyl, 3-pyridyl, 4-pyridyl, 2-pyrimidyl, 4-pyrimidyl, 2-pyranyl, 3-pyranyl, 3-pyrazolyl, 4-pyrazolyl, 5-pyrazolyl, 2-pyrazinyl, 2-thiazolyl, 4-thiazolyl, 5-thiazolyl, 2-oxazolyl, 4-oxazolyl and 5-oxazolyl.

Aryl groups also include fused polycyclic aromatic ring systems in which a carbocyclic, alicyclic, or aromatic ring or heteroaryl ring is fused to one or more other heteroaryl or aryl rings. Examples include 2-benzothienyl, 3-benzothienyl, 2-benzofuranyl, 3-benzofuranyl, 2-indolyl, 3-indolyl, 2-quinolinyl, 3-quinolinyl, 2-benzothiazole, 2-benzooxazole, 2-benzimidazole, 2-quinolinyl, 3-quinolinyl, 1-isoquinolinyl, 3-quinolinyl, 1-isoindolyl and 3-isoindolyl.

It will be understood that salts of the compounds described above, where the compounds have one or more basic moieties, also comprise part of the present invention. Suitable acids for making such salts include hydrochloric, sulfuric or phosphoric acids, as well as methanesulfonic, arginine, lysine, and the like.

The invention also includes pharmaceutically acceptable salts of the carboxylic acid moieties of the compounds described above. Pharmaceutically acceptable salts include ammonium salts and metal salts such as the alkali metal and alkaline earth metals salts, e.g., sodium, potassium, magnesium or calcium salts, as well as divalent metal salts such as zinc. Pharmaceutically acceptable salts include salts with suitable organic amines, such as aliphatic, cycloaliphatic, cycloaliphatic-aliphatic or araliphatic primary, secondary or tertiary mono-, di- or poly-amines, and also heterocyclic bases. Examples of such amines include lower alkylamines (e.g., triethylamine), hydroxy-lower alkylamines (e.g., 2-hydroxyethylamine, bis-(2-hydroxyethyl)-amine, tris-(2-hydroxyethyl)-amine), basic aliphatic esters of carboxylic acids, (e.g., 4-aminobenzoic acid 2-diethylaminoethyl ester), lower alkyleneamines (e.g., 1-ethylpiperidine), cycloalkylamines (e.g., dicyclohexylamine), benzylamines (e.g., N,N′-dibenzylethylenediamine), and bases of the pyridine type (e.g., pyridine, collidine, quinoline). Further salts include internal salts (zwitterionic forms of compounds of the invention), wherein a basic group, for example, a basic nitrogen atom present in a pyridine ring, is protonated by a hydrogen ion originating from an acid group in the molecule.

Compounds of the invention have the ability to suppress the formation of free radicals and other oxidant species. In one example, radical formation is suppressed by stabilizing iron(III). Compounds of the present invention have been shown to stabilize iron(III) when the compound to iron ratio is about 0.25 or greater or about 0.5 or greater. This is an unexpected feature of these compounds, as Example 1 demonstrates that other compounds that interact with iron(III) such as nitrilotriacetic acid, 5-aminosalicylic acid, 1,2-dimethyl-3-hydroxypyridin-4-one, and N-hydroxy,N-(3,6,9-trioxadecyl)acetamide (hereinafter “decyl hydroxamate”) increased the rate of iron(III) reduction in the presence of ascorbate when the ratio of compound to iron was 0.5 to 3.0. In contrast, compounds of the present invention decreased the rate of iron(I1I) reduction at all ratios of compound to metal analyzed. Suitable ratios of a compound of the present invention to metal include about 0.25 to about 10 or more, about 0.25 to about 5.0, about 0.5 to about 3.0, about 0.5 to about 2.0, and about 1.0 to about 2.0.

If iron(III) is present in a system, it is advantageously present in combination with a compound of the present invention, thereby stabilizing iron(III). Iron(III) is advantageously stabilized when it can be in contact with hydrogen peroxide, an organic peroxide, or a nitrosothiol. In these situations, iron(II) can react with hydrogen peroxide or an organic peroxide to form a damaging hydroxyl or alkoxyl (e.g., RO where R is an alkyl group) radical. Iron(II) can also react with a nitrosothiol to form nitric oxide. Nitric oxide can react with superoxide anion at a diffusion-controlled rate to form peroxynitrite, a potent and damaging oxidizing agent. This stabilization of iron(III) can occur in patients not suffering from an excess body burden of iron, i.e., patients with normal or low iron levels. Also, iron(III) can be stabilized in vitro, including in consumer products subject to oxidation or degradation by free radicals. In one embodiment of the invention, stabilizing iron(III) does not prevent peroxidation of a substance.

When preventing or inhibiting oxidation of a substance, compounds of the present invention can be contacted with a substance in vivo or in vitro, but preferably in vitro. Suitable substances include food products and other organic compounds that can react with free radicals. Food products suitably contacted with one or more compounds of the present invention include vitamins or foods with high lipid content (e.g., greater than 20% lipid by weight, greater than 40% lipid by weight, greater than 60% lipid by weight), foods whose flavor is diminished or affected by reaction with free radicals, and foods that are stored for long periods (e.g., more than one week, more than one month, more than six months, or more than one year) prior to consumption. Such food products include those comprising vegetable fat, lard, butter, mayonnaise, egg yolks, potato chips, corn chips, chocolate, bacon, beef, pork, lamb, other meats, milk, cream, self-stabilized foods, and food for consumption by military personnel (e.g., meals-ready-to-eat). Other products that benefit from the presence of a substance of the invention include shampoos, hair conditioners, hair styling products, and cosmetics. A substance treated in vitro with one or more compounds of the present invention is typically in contact with reduced metal ions (e.g., Fe(II) or Cu(l)), sunlight, hydrogen peroxide, superoxide, organic peroxides, nitrosothiols, or a combination thereof. Such substances often contain oxidizable moieties, such as unsaturated carbon-carbon bonds (e.g., double or triple bonds, particularly conjugated unsaturated bonds), aldehydes, epoxides, amines, azo groups, azido groups, thiols, sulfenic acid, sulfinic acid, phosphines, and nitriles.

A patient in need of antioxidant therapy can have one or more of the following conditions: decreased levels of reducing agents, increased levels of reactive oxygen species, mutations in or decreased levels of antioxidant enzymes (e.g., Cu/Zn superoxide dismutase, Mn superoxide dismutase, glutathione reductase, glutathione peroxidase, thioredoxin, thioredoxin peroxidase, DT-diaphorase), mutations in or decreased levels of dicationic transition metal-binding proteins, mutated or overactive enzymes capable of producing superoxide (e.g., nitric oxide synthase, NADPH oxidases, xanthine oxidase, NADH oxidase, aldehyde oxidase, dihydroorotate dehydrogenase, cytochrome c oxidase), and radiation injury. Increased or decreased levels of reducing agents, reactive oxygen species, and proteins are determined relative to the amount of such substances typically found in healthy persons. In one embodiment, the patient has normal (non-mutated) metal binding proteins present in sufficient quantities and/or normal iron levels.

A patient who is advantageously treated to suppress formation of radical species typically has increased levels of reducing agents (especially superoxide, ascorbate, or glutathione), reduced levels of dicationic transition metal-binding proteins, increased levels of hydrogen peroxide or organic peroxides, increased levels of nitrosothiols, or a combination of the above conditions. In some embodiments of the invention, the patient has a normal or decreased body burden of iron, normal or increased levels of metal-binding proteins, or both. In particular, in some embodiments of the invention, the patient being treated has normal iron levels, e.g., does not suffer from hemochromatosis, hemosiderosis or cirrhosis.

Reducing agents include vitamin A and related compounds such as 0-carotene; vitamin C (ascorbic acid); vitamin E and related compounds such as a-tocopherol; cysteine; glutathione; N-acetylcysteine; mecaptopropionylglycine; uric acid; ubiquinol; bilirubin; and selenium.

Reactive oxygen species include superoxide, hydrogen peroxide, organic peroxides, singlet oxygen, ozone, hypochlorous acid (HOCI), thiyl radical, nitric oxide, nitrogen dioxide, ferryl complexes (i.e., containing Fe(IV)═O), and free radicals such as hydroxyl radical, organic hydroxyl radical (e.g., lipid hydroxyl radical, alkoxyl radical, alkenoxyl radical), hydrogen peroxyl radical, and organic peroxyl radical (e.g., a lipid peroxyl radical). An organic peroxide is of the formula R′OOH, where R′ is a substituted or unsubstituted alkyl group. Similarly, an organic peroxyl radical is of the formula R′OO and an organic hydroxyl radical is of the formula R′O , where R′ is as defined above.

Free radicals also include organic radicals (e.g., carbon-centered radicals, nitrogen-center radicals, sulfur-centered radicals, oxygen-centered radicals) such as lipids and other molecules containing double or triple carbon-carbon bonds (e.g., tocopherol (vitamin E) and beta-carotene (vitamin A)). Compounds disclosed herein are effective both in quenching free radicals and in terminating chain propagation reactions, such as the reaction of a lipid radical with oxygen.

Ischemic episodes can occur when there is local anemia due to mechanical obstruction of the blood supply, such as from arterial narrowing or disruption. Myocardial ischemia, which can give rise to angina pectoris and myocardial infarctions, results from inadequate circulation of blood to the myocardium, usually due to coronary artery disease. Ischemic episodes in the brain that resolve within 24 hours are referred to as transient ischemic attacks. A longer-lasting ischemic episode, a stroke, involves irreversible brain damage, where the type and severity of symptoms depend on the location and extent of brain tissue whose circulation has been compromised. A patient at risk of suffering from an ischemic episode typically suffers from atherosclerosis, other disorders of the blood vessels, increased tendency of blood to clot, or heart disease. The compounds of this invention can be used to treat these disorders. In one embodiment of the invention, the patient suffering from or at risk of suffering from ischemic episodes does not suffer from a trivalent metal (e.g., iron) overload.

Inflammation is a fundamental pathologic process consisting of a complex of cytologic and chemical reactions that occur in blood vessels and adjacent tissues in response to an injury or abnormal stimulation caused by a physical, chemical, or biologic agent. Inflammatory disorders are characterized by inflammation that lasts for an extended period (i.e., chronic inflammation) or that damages tissue. Markers of inflammatory disease include chronically increased levels of cycloxygenase-2, histamine and/or cytokines, as well as an extended elevation of leukocyte levels. Such inflammatory disorders can affect a wide variety of tissues, such as respiratory tract, vasculature, joints, and soft tissue. The compounds of this invention can be used to treat these disorders.

Specific inflammatory disorders contemplated by this invention include gout, arthritis (rheumatoid arthritis), asthma, atherosclerosis, hyperproliferative anemia, megaloblastic anemia, disorders resulting from chronic infection (pelvic inflammatory disorder, tissue/organ damage associated with cystic fibrosis), psoriasis, allergic inflammation, atopic dermatis (eczema), ocular inflammatory diseases (uveitis, scleritis, episcleritis, age-related macular degeneration), celiac disease, hypereosinophilic syndrome, ankylosing spondylitis, bursitis, chronic obstructive pulmonary disease and allergic rhinitis. In addition, many autoimmune disorders are believed to be inflammatory disorders. Autoimmune disorders include Hashimoto's thyroiditis, Graves' disease, type I autoimmune polyglandular syndrome, type II autoimmune polyglandular syndrome, insulin-dependent diabetes mellitus, immune-mediated infertility, pemphigus vulgaris, pemphigus foliaceus, paraneoplastic pemphigus, bullus pemphigoid, dermatitis herpetiformis, linear IgA disease, epidermolysis bullosa acquisita, autoimmune alopecia, erythema nodosa, pemphigoid gestationis, cicatricial pemphigoid, chronic bullous disease, autoimmune hemolytic anemia, autoimmune thrombocytopenic purpura, autoimmune neutropenia, myasthenia gravis, Eaton-Lambert myasthenic syndrome, stiff-man syndrome, acute disseminated encephalomyelitis, multiple sclerosis, Guillain-Barré syndrome, chronic inflammatory demyelinating polyradiculoneuropathy, multifunctional motor neuropathy, IgA chronic neuropathy with monoclonal gammopathy, opsoclonus-myoclonus syndrome, cerebellar degeneration, encephalomyelitis, retinopathy, autoimmune chronic active hepatitis, primary biliary sclerosis, sclerosing cholangitis, gluten-sensitive enteropathy, pernicious anemia, systemic lupus erythematosus, rheumatory arthritis, systemic sclerosis (scleroderma), reactive arthritides, polymyositis, dermatomyositis, Sjögrens' syndrome, mixed connective tissue disease, Behcet's syndrome, psoriasis, systemic necrotizing vasculitides, hypersensitivity vasculitis, Wegener's granulomatosis, temporal arteritis, Takayasu's arteritis, Kawasaki's disease, isolated vasculitis of the central nervous system, thromboangiitis obliterans, sarcoidosis, IgA nephropathy, Addison disease, Goodpasture syndrome, poststreptococcal glomerulonephritis, graft-versus-host disease and cryopathies. The compounds of this invention can be used to treat these disorders. In one embodiment of the invention, autoimmune and inflammatory disease exclude inflammatory bowel disease. In another embodiment, the chronic infection causing inflammation is not a Plasmodium species. In yet another embodiment of the invention, the patient suffering from the inflammatory disorder has normal or low levels of iron.

Neoplastic disease is characterized by an abnormal tissue that grows by cellular proliferation more rapidly than normal tissue. The abnormal tissue continues to grow after the stimuli that initiated the new growth cease. Neoplasms show a partial or complete lack of structural organization and functional coordination with the normal tissue, and usually form a distinct mass of tissue that may be either benign or malignant. Neoplasms can occur, for example, in a wide variety of tissues including brain, skin, mouth, nose, esophagus, lungs, stomach, pancreas, liver, bladder, ovary, uterus, testicles, colon, and bone, as well as the immune system (lymph nodes) and endocrine system (thyroid gland, parathyroid glands, adrenal gland, thymus, pituitary gland, pineal gland). The compounds of this invention can be used to treat these disorders.

A preneoplastic condition precedes the formation of a benign or malignant neoplasm. A precancerous lesion typically forms before a malignant neoplasm. Preneoplasms include photodermatitis, x-ray dermatitis, tar dermatitis, arsenic dermatitis, lupus dermatitis, senile keratosis, Paget disease, condylomata, burn scar, syphilitic scar, fistula scar, ulcus cruris scar, chronic ulcer, varicose ulcer, bone fistula, rectal fistula, Barrett esophagus, gastric ulcer, gastritis, cholelithiasis, kraurosis vulvae, nevus pigmentosus, Bowen dermatosis, xeroderma pigmentosum, erythroplasia, leukoplakia, Paget disease of bone, exostoses, ecchondroma, osteitis fibrosa, leontiasis ossea, neurofibromatosis, polyposis, hydatidiform mole, adenomatous hyperplasia, and struma nodosa. The compounds of this invention can be used to treat these disorders.

In one embodiment of the present invention, the disease or condition being treated is not neoplastic. In addition, certain embodiments of the invention exclude neoplastic or pre-neoplastic conditions caused by iron overload.

In another embodiment of the invention, the disease or condition being treated is not associated with dialysis, such as encephopathy and osteomalacia (e.g., from aluminum toxicity).

Compounds of the present invention can also be used to treat patients suffering from neurodegenerative diseases, and traumatic or mechanical injury to the central nervous system (CNS). Neurodegenerative disease typically involves reductions in the mass and volume of the human brain, which may be due to the atrophy and/or death of brain cells, which are far more profound than those in a healthy person that are attributable to aging. Neurodegenerative diseases evolve gradually, after a long period of normal brain function, due to progressive degeneration (e.g., nerve cell dysfunction and death) of specific brain regions. The actual onset of brain degeneration may precede clinical expression by many years. For example, clinical manifestations of parkinsonism become apparent following a loss of ˜80% of nigral dopaminergic neurons (i.e., nerve cells involved in motor behavior), and this may occur over several years. Examples of neurodegenerative diseases include Alzheimer's disease, Parkinson's disease, Huntington disease, amyotrophic lateral sclerosis (Lou Gehrig's disease), diffuse Lewy body disease, chorea-acanthocytosis, primary lateral sclerosis, and Friedreich's ataxia. The compounds of this invention can be used to treat these disorders. In one embodiment of the invention, neurodegenerative diseases exclude Alzheimer's disease. In another embodiment of the invention, neurodegenerative disease exclude those caused by an excess of trivalent metals (e.g., aluminum, iron).

In a preferred embodiment of the invention, the disease or condition being treated does not result from an excess of a trivalent metal ion, especially iron. The excess of the trivalent metal ion can be global, focal (i.e., limited to a specific group of cells or tissues) or both.

As a method of treatment, a compound of the present invention can retard the progression, reduce symptoms, reduce biological damage, inhibit the onset of symptoms or biological damage, or inhibit relapse or recurrence of a disease, disorder, or condition.

The compounds of this invention can be administered as the sole active ingredient or in combination with other active agents.

The compounds or pharmaceutically acceptable salts thereof of the present invention in the described dosages are administered orally, intraperitoneally, subcutaneously, intramuscularly, transdermally, sublingually or intravenously.

They are preferably administered orally, for example, in the form of tablets, troches, capsules, elixirs, suspensions, syrups, wafers, chewing gum or the like prepared by art recognized procedures. The amount of active compound in such therapeutically useful compositions or preparations is such that a suitable dosage will be obtained.

The pharmaceutical compositions of the invention preferably contain a pharmaceutically acceptable carrier or excipient suitable for rendering the compound or mixture administrable orally as a tablet, capsule or pill, or parenterally, intravenously, intradermally, intramuscularly or subcutaneously, rectally, via inhalation or via buccal administration, or transdermally. The active ingredients may be admixed or compounded with any conventional, pharmaceutically acceptable carrier or excipient. It will be understood by those skilled in the art that any mode of administration, vehicle or carrier conventionally employed and which is inert with respect to the active agent may be utilized for preparing and administering the pharmaceutical compositions of the present invention. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention.

The therapeutically effective amount of active agent to be included in the pharmaceutical composition of the invention depends, in each case, upon several factors, e.g., the type, size and condition of the patient to be treated, the intended mode of administration, the capacity of the patient to incorporate the intended dosage form, etc. Active agents serving as an antioxidant or free radical scavenger preferably inactivate at least about 10%, at least about 20%, at least about 30%, at least about 40%, at least about 50%, at least about 60%, at least about 70%, at least about 80% or at least about 90% of the targeted oxidant or free radical, either generally or in a specific tissue or organ. Alternatively, active agents of the invention reduce oxidant or free radical levels such that a patient's own mechanisms for neutralizing such species are able to reduce oxidant or free radical levels such that no additional biological damage (macroscopic or microscopic) can be measured or that the rate at which biological damage occurs is significantly reduced.

A therapeutically effect amount of a compound used to treat a disease, disorder, or condition disclosed herein is an amount sufficient to retard the progression, reduce symptoms, reduce biological damage, inhibit the onset of symptoms or biological damage, or inhibit relapse or recurrence of the disease, disorder, or condition.

Typical daily dosages of active agents (e.g., for a 70 kg patient) used in the methods of the instant invention range from about 10 mg to about 10,000 mg, preferably 50 mg to about 10,000 mg, and more preferably about 300 mg to about 1000 mg. The dose can be administered in one, two, three, four, six, eight, ten, twelve or more portions during the day, or can be delivered continuously via an intravenous line or via an external or implanted pump.

While it is possible for the agents to be administered as the raw substances, it is preferable, in view of their potency, to present them as a pharmaceutical formulation. The formulations of the present invention for human use comprise the agent, together with one or more acceptable carriers therefor and optionally other therapeutic ingredients. The carrier(s) must be “acceptable” in the sense of being compatible with the other ingredients of the formulation and not deleterious to the recipient thereof. Desirably, the formulations should not include oxidizing agents and other substances with which the agents are known to be incompatible. The formulations may conveniently be presented in unit dosage form and may be prepared by any of the methods well known in the art of pharmacy. All methods include the step of bringing into association the agent with the carrier that constitutes one or more accessory ingredients. In general, the formulations are prepared by uniformly and intimately bringing into association the agent with the carrier(s) and then, if necessary, dividing the product into unit dosages thereof.

Formulations suitable for parenteral administration conveniently comprise sterile aqueous preparations of the agents which are preferably isotonic with the blood of the recipient. Suitable such carrier solutions include phosphate buffered saline, saline, water, lactated ringers or dextrose (5% in water). Such formulations may be conveniently prepared by admixing the agent with water to produce a solution or suspension which is filled into a sterile container and sealed against bacterial contamination. Preferably, sterile materials are used under aseptic manufacturing conditions to avoid the need for terminal sterilization.

Such formulations may optionally contain one or more additional ingredients among which may be mentioned preservatives, such as methyl hydroxybenzoate, chlorocresol, metacresol, phenol and benzalkonium chloride. Such materials are of special value when the formulations are presented in multidose containers.

Buffers may also be included to provide a suitable pH value for the formulation. Suitable such materials include sodium phosphate and acetate. Sodium chloride or glycerin may be used to render a formulation isotonic with the blood. If desired, the formulation may be filled into the containers under an inert atmosphere such as nitrogen and are conveniently presented in unit dose or multi-dose form, for example, in a sealed ampoule.

Those skilled in the art will be aware that the amounts of the various components of the compositions of the invention to be administered in accordance with the method of the invention to a patient will depend upon those factors noted above.

The compositions of the invention when given orally or via buccal administration may be formulated as syrups, tablets, capsules and lozenges. A syrup formulation will generally consist of a suspension or solution of the compound or salt in a liquid carrier, for example, ethanol, glycerine or water, with a flavoring or coloring agent. Where the composition is in the form of a tablet, any pharmaceutical carrier routinely used for preparing solid formulations may be employed. Examples of such carriers include magnesium stearate, starch, lactose and sucrose. Where the composition is in the form of a capsule, any routine encapsulation is suitable, for example, using the aforementioned carriers in a hard gelatin capsule shell. Where the composition is in the form of a soft gelatin shell capsule, any pharmaceutical carrier routinely use for preparing dispersions or suspensions may be considered, for example, aqueous gums, celluloses, silicates or oils, and are incorporated in a soft gelatin capsule shell.

A typical suppository formulation comprises the active agent or a pharmaceutically acceptable salt thereof which is active when administered in this way, with a binding and/or lubricating agent, for example, polymeric glycols, gelatins, cocoa-butter or other low melting vegetable waxes or fats.

Typical transdermal formulations comprise a conventional aqueous or non-aqueous vehicle, for example, a cream, ointment, lotion or paste or are in the form of a medicated plastic, patch or membrane.

Typical compositions for inhalation are in the form of a solution, suspension or emulsion that may be administered in the form of an aerosol using a conventional propellant such as dichlorodifluoromethane or trichlorofluoromethane.

Exemplification

### EXAMPLE 1

Prevention of Iron-Mediated Oxidation of Ascorbate.

Compounds were tested for their ability to diminish the iron-mediated oxidation of ascorbate by the method of Dean and Nicholson (Free Radical Res. 20, 83-101 (1994)). Briefly, a solution of freshly prepared ascorbate (100 μM) in sodium phosphate buffer (5 mM, pH 7.4) was incubated in the presence of FeCl₃ (30 μM) and the test compound (compound/Fe ratios varied from 0-3) for 40 min. The A₂₆₅ was read at 10 and 40 min; the ΔA₂₆₅ in the presence of the compound was compared to that in its absence.

Desferrioxamine B in the form of the methanesulfonate salt, Desferal (Novartis Pharma AG, Basel, Switzerland), was obtained from a hospital pharmacy. 1,2-Dimethyl-3-hydroxypyridin-4-one (L1) was a generous gift from Dr. H. H. Peter (Ciba-Geigy, Basel).

Spectrophotometric readings (A_{λ}) for the ascorbate and radical cation assays were taken on a Perkin-Elmer Lambda 3B spectrophotometer (Norwalk, Conn.).

The role of compounds in suppressing or enhancing radical formation, e.g., either inhibition or promotion of the Fenton reaction, is related to their capacity to prevent Fe(III) from being reduced to Fe(II). Fe(II) is required for the reduction of H₂O₂ to HO• and HO⁻. The assay involves spectrophotometrically monitoring the disappearance of ascorbate at pH 7.4 in the presence of FeCl₃ and a compound at several compound/Fe ratios. Under these conditions, ascorbate is oxidized to an L-ascorbyl radical anion. This anion then disproportionates to dehydroascorbic acid and ascorbate.

Some compounds [e.g., the hydroxypyridinone 1,2-dimethyl-3-hydroxypyridin-4-one (L1)] began to prevent ascorbate reduction of Fe(III) at compound:metal ratios of 3: 1, but below this ratio, reduction was actually stimulated. This was also true with another compound, 5-aminosalicylic acid (5-ASA), the active ingredient in Rowasa®, one of the currently accepted therapeutic agents for inflammatory bowel disease (IBD). Nitrilotriacetic acid (NTA) dramatically stimulated Fe(III) reduction. The parameters that control whether a compound promotes Fe(III) reduction at a given compound:metal ratio are quite complicated.

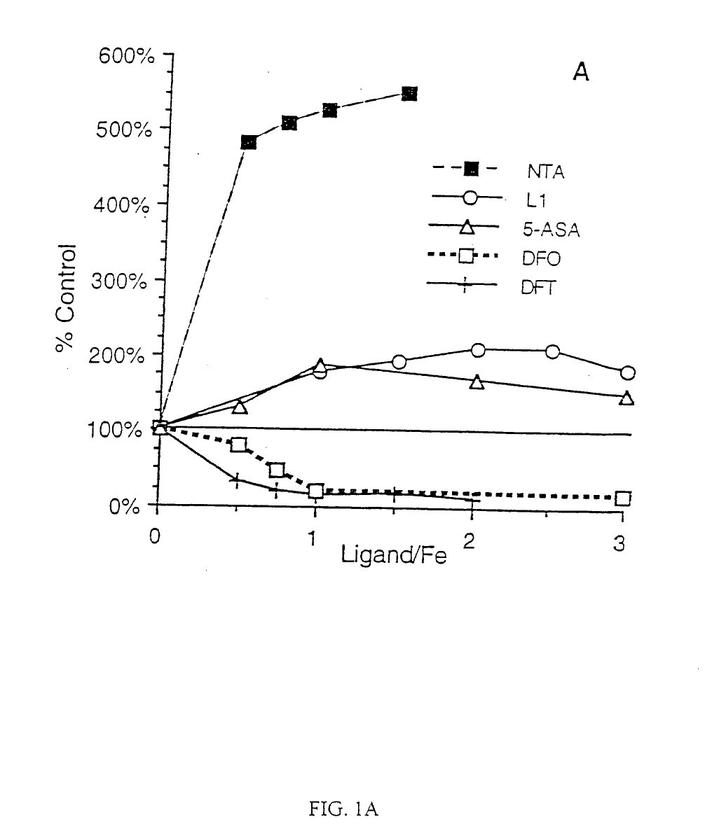

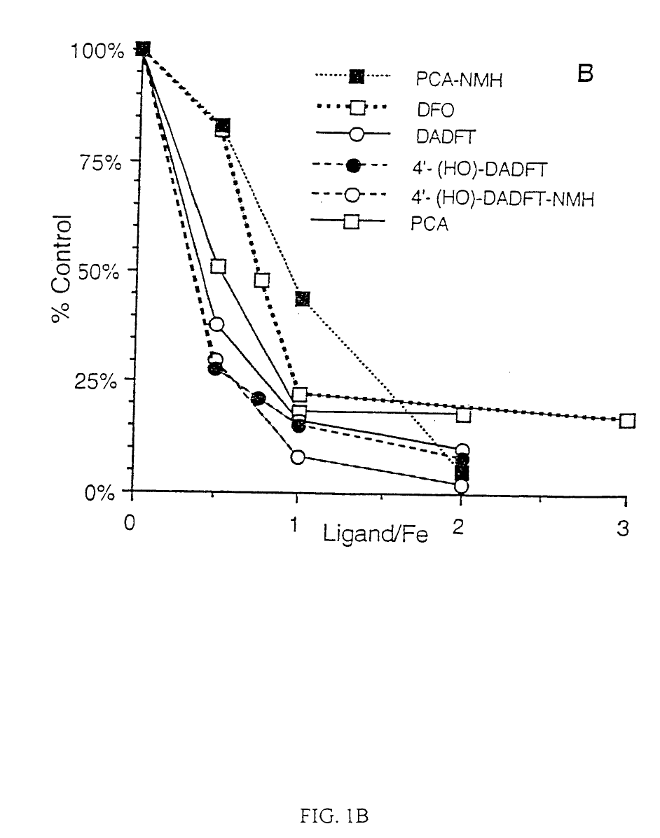

In the current study, four control compounds were evaluated (FIG. 1A), along with several desferrithiocin analog carboxylic acids and their corresponding hydroxamates (representative selection, FIG. 1B) for their ability to affect ascorbate reduction of Fe(III). Consistent with previous findings, NTA, L1, and 5-ASA promoted ascorbate-mediated reduction of Fe(III), even at a compound:metal ratio of 3:1. However, the stimulation mediated by both L1 and 5-ASA was beginning to diminish at this ratio. The significant inhibition of the reaction by DFO in the present experiment was also in keeping with the observations in the literature.

How these compounds affect the rate of this reaction is interesting; of particular significance is that none of the desferrithiocin analogs, neither carboxylic acid nor hydroxamate derivatives, stimulated ascorbate-mediated Fe(III) reduction. This is true even at compound:metal ratios of 0.5:1 (FIG. 1B). In fact, all of the compounds were protective. Most intriguing is the fact that, with the exception of the N-methylhydroxamate of PCA (3,4-dihydro-5-(2-hydroxy-5-methylphenyl)-2H-pyrrole-2-carboxylic acid), all of the analogs were more effective than desferrioxamine at all of the compound:metal ratios tested. It is quite clear that, as a family, the desferrithiocin analogs do inhibit ascorbate-mediated reduction of Fe(III). It is interesting that although L1 potentiated iron-mediated oxidative DNA damage in iron-loaded hepatocytes, desferrithiocin (DFT) prevented damage in this model.

### EXAMPLE 2

Quenching of the ABTS Radical Cation

Compounds were tested for their ability to quench the radical cation formed from 2,2′-azinobis(3-ethylbenzothiazoline-6-sulfonic acid) (ABTS) by the method of Re et al. in Free Radic. Biol. Med. 26, 1231-1237 (1999). Briefly, a stock solution of ABTS radical cation was generated by mixing ABTS (10 mM, 2.10 mL) with K₂S₂O₈ (8.17 mM, 0.90 mL) in H₂O and allowing the solution of deep blue-green ABTS radical cation was diluted in sufficient sodium phosphate (10 mM, pH 7.4) to give an A₇₃₄ of about 0.900. Test compounds were added to a final concentration ranging from 1.25 to 15 μM, and the decrease in A₇₃₄ was read after 1, 2, 4 and 6 min. The reaction was largely complete by 1 min, but the data presented are based on a 6-min reaction time.

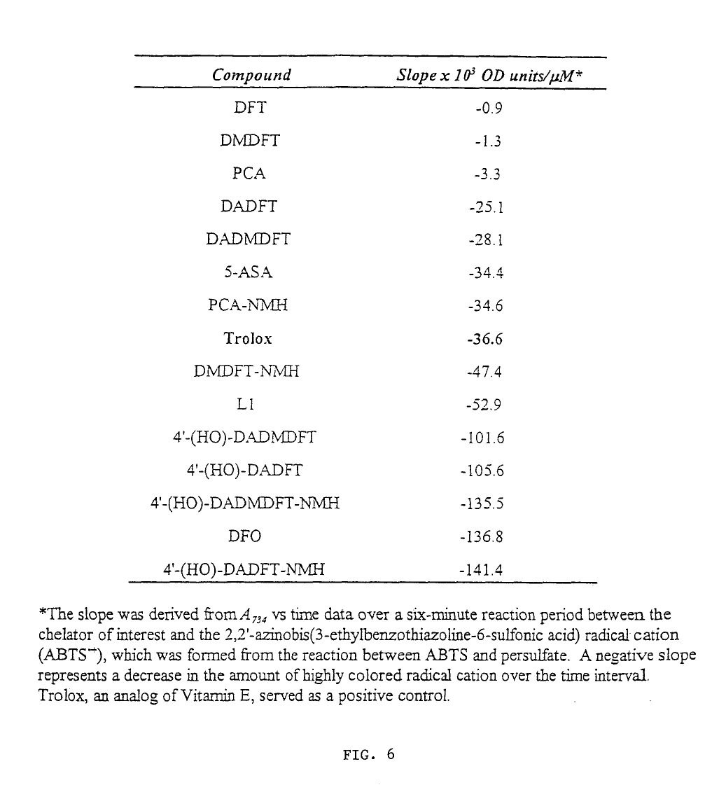

In this assay, a fairly stable radical cation, ABTS⁺, was examined, and Trolox, an analog of vitamin E, was used as a positive control. Briefly, the procedure involved generating the blue-green chromophore by the reaction of ABTS with K₂S₂O₈. The radical has absorption maxima at λ=415, 645, 734, and 815 nm. The change in absorbance at 734 nm was noted 6 min after addition of the compound of interest at various concentrations, and the slope of the ΔA₇₃₄ vs. compound concentration line was calculated. These slopes are shown in FIG. 6.

When the radical scavenging abilities of the desferrithiocin carboxylic acids and their hydroxamates are compared, there are several notable observations. First, the hydroxamates are always more effective scavengers than are the corresponding free acids, (e.g., desmethyldesferrithiocin—N-methyl-hydroxamate (DMDFT—NMH) vs. desmethyldesferrithiocin (DMDFT)). Removal of the aromatic nitrogen from desferrithiocin substantially increased radical scavenging capacity. Introduction of a 4′-hydroxyl also considerably enhanced radical scavenging properties, such as 4′-hydroxydesazadesmethyldesferrithiocin (4′-(HO)-DADMDFT) vs. desazadesmethyldesferrithiocin (DADMDFT). Trolox, the positive control, and 5-ASA are very similar in their scavenging properties, though slightly less effective than L1. Desferrioxamine, the 4′-hydroxylated desferrithiocin analogs and their corresponding hydroxamates were the most effective scavenging agents.

### EXAMPLE 3

Rodent Model of Acid-Induced Colitis and Inflammatory Bowel Disease (IBD)

Drug Preparation and Administration. Compounds were administered to the rats intracolonically as a suspension or solution in distilled water (2 mL) at a dose of 650 μmol kg⁻¹. The drug solutions were made fresh for each experiment. Rowasa®, the pharmaceutical preparation which contains 5-ASA (2 mL, 66.7 mg mL⁻¹) was given at a dose of 2318 μmol kg⁻¹. Control rats received distilled water (2 mL), administered intracolonically.

Induction of Colitis. Animal care and experimental procedures were approved by the Institutional Animal Care and Use Committee. Colitis was induced by a modification of published methods. Briefly, the rats were anesthetized with sodium pentobarbital, 55 mg kg⁻¹ intraperitoneally. The abdomen was shaved and prepared for surgery. A midline incision was made, and the cecum and proximal colon were exteriorized. A reversible suture was placed at the junction of the cecum and proximal colon. The colon was rinsed with saline (10 mL), and the fluid and intestinal contents were gently expressed out the rectum. A gum-based rectal plug was inserted. The compound of interest, or distilled water in the control animals (2 mL), was injected intracolonically just distal to the ligature. The cecum and proximal colon were returned to the abdominal cavity; the compound was allowed to remain in the gut for 30 min. Then, the cecum and proximal colon were reexteriorized. The rectal plug was removed, and the drug was gently expressed out of the colon. Acetic acid (4%, 2 mL) was injected into the proximal colon over a 15-20-second time period. The acid was allowed to remain in the gut until one minute had passed (i.e., 40-45 seconds after the end of the acid administration). The no acid control rats received distilled water (2 mL), which was administered in the same manner as was the acetic acid. Air (10 mL) was then injected into the proximal colon to expel the acid or water. The cecal/proximal colon ligature was removed, the gut was returned to the abdominal cavity, and the incisions were closed. The animals were allowed to recover overnight and were sacrificed 24 hr later. The entire length of the colon was removed and assessed for damage both densitometrically and biochemically.

Quantification of Acetic Acid-Induced Colitis. Gross damage was quantitated using Photoshop-based image analysis (version 5.0, Adobe Systems, Mountain View, Calif., USA) on an Apple iMac computer. The Magic Wand tool in the Select menu of Photoshop was used to place the cursor on an area of obvious damage. The tolerance level of the Magic Wand tool was set at 30. The damaged areas were automatically selected by using the Similar command in the Select menu. Then, the Eyedropper tool was used to determine the range of the damage in the highlighted areas. Individual colon images were copied to a blank Photoshop page. The Magic Wand tool, with a tolerance set to 100, was used to select all of the pixels in the colon sample. Then, the Histogram tool, which generates a graph in which each vertical line represents the number of pixels associated with a brightness level, was selected in the Image menu. The Red channel was then selected; the darker (damaged areas) appear on the left side of the histogram and the lighter (normal) areas are on the right side. The cursor was then placed on the histogram, the color range determined in an earlier step was selected, and the number of pixels encompassing that range and the percent damage were quantified automatically.

Myeloperoxidase (MPO) Assay. The activity of MPO was measured in colonic tissue by a modification of the method of Krawisz et al. in Gastroenterology 87, 1344-1350 (1984). Each excised colon was homogenized in 9 volumes of homogenization buffer (0.5% hexadecyltrimethylammonium bromide (HDTMA) in 50 mM sodium acetate, pH 6.0); this homogenate was centrifuged at 1200g for 20 min at 4° C. A sample of the supernatant (1.8 mL) was transferred into a microcentriflge tube and stored frozen at −20° C. for up to one week. Prior to assay, the thawed aliquot was centrifuged at about 10,000 g for 15 minutes at 4° C. The final supernatant (33 μL) was added to a solution of o-dianisidine HCl (0.17 mg mL⁻¹ in 50 mM sodium acetate, pH 6.0, made fresh daily and filtered immediately before use) (950 μL), the mixture was vortexed, and the peroxidase reaction was initiated by the addition of H₂O₂ to 50 mM sodium acetate, pH 6.0, made fresh daily (16.7 μL). The A₄₇₀ at room temperature (ca. 23° C.) was read at 15-second intervals for 2 min. The rates were assessed graphically and are presented as change in milliabsorbance units (ΔmAU)/min per g tissue. Under these conditions, 0.1 “Unit” of purified human leukocyte myeloperoxidase (Sigma M-6908) produced a ΔA₄₇₀ of about 400 mAU/min.

IBD Rodent Model. The acetic acid-induced model of IBD is particularly attractive for rapid screening. Exposure of the rat colon to acetic acid elicits diffuse hemorrhagic necrosis with significant erosion of microvascular mucosal barriers as measured by ⁵¹Cr-labeled erythrocyte clearance into the lumen.

Two means were employed to assess the damage to the colon in the presence and absence of compound, computer-based image analysis and colonic MPO measurement. The densitometric method removes much of the subjectivity involved in the simple scoring approaches. A digital image of the prepared colonic tissue is taken, and a clearly damaged segment is highlighted on the screen. Once the computer identifies all other segments of the intestine with the same or greater damage, a pixel number is generated; this number makes it possible to calculate the percentage of damaged intestine.

The biochemical measurement involves measuring the level of MPO in a sample of homogenate of the whole rat colon. When the colon is damaged by the acetic acid, there is an extravasation of neutrophils. The extent of this infiltration can serve as a quantitative marker for tissue damage. Although other leukocytes, such as eosinophils and monocytes, also contribute to the inflammatory response, their contribution is small; the majority of the cells recruited during the acute inflammatory response are neutrophils. Thus, the MPO assay serves as an “index of neutrophil infiltration”. Because the neutrophil granules contain as much as 5% MPO, the assay is particularly sensitive for these phagocytes. Briefly, the assay involved homogenization of the entire rat colon and centrifugation to remove tissue and cellular debris. The supernatant is combined with an indicator and H₂O₂, and the reaction is monitored spectrophotometrically.

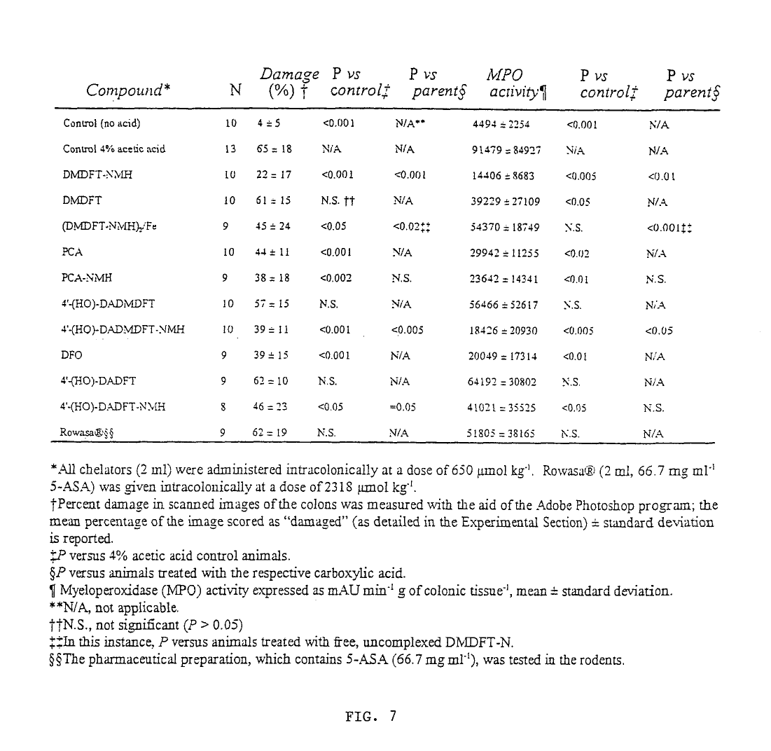

The results in FIG. 7 are arranged such that the damage calculated densitometrically and biochemically appear together. The desferrithiocin analogs are presented in four sets; the hydroxamate is paired with the parent carboxylic acid. In one instance, the iron complex of DMDFT—NMH was also evaluated. Finally, Rowasa®, the active ingredient of which is 5-ASA, was tested along with controls treated with acetic acid and no compound and naive controls. P-Values were calculated between each of the compounds and acetic acid treated controls and, where applicable, between the hydroxamates and their respective parent carboxylic acids.

Of all of the analogs tested, the most effective was the hydroxamate DMDFT-NMH. Animals treated with this compound sustained significantly less damage than acetic acid-treated controls, as measured both densitometrically (P<0.001) and biochemically (P<0.005). The biochemical assay suggested that this compound's parent, DMDFT, was also effective (P<0.05). Clearly, the hydroxamate DMDFT-NMH was better than its parent carboxylic acid DMDFT (P<0.001 by densitometry, P<0.01 by MPO assay). The colons of animals treated with the iron complex of DMDFT—NMH appeared to be damaged more than those from animals treated with the uncomplexed compound (P<0.05 vs control; P<0.02 vs DMDFT—NMH by image analysis, and P N.S. vs. control; P<0.001 vs DMDFT—NMH by MPO assay).

Animals treated with the N-methylhydroxamate of PCA (PCA—NMH) also fared better than did acetic acid controls (P<0.002 and P<0.01 by image analysis and biochemistry, respectively), as did animals treated with the parent carboxylic acid, PCA (P<0.001 and P<0.02 by image analysis and MPO assay, respectively). The difference between two analogs, however, was not significant (P<0.05 by both measurements). There was a striking difference between 4′-(HO)-DADMDFT and its N-methylhydroxamate (P<0.005 and P<0.05 by densitometry and biochemistry, respectively). Although the carboxylic acid was ineffective (P>0.05 by both measurements), the hydroxamate derivative significantly protected the rats from acetic acid-induced colonic damage (P<0.001 and P<0.005 by image analysis and MPO, respectively).

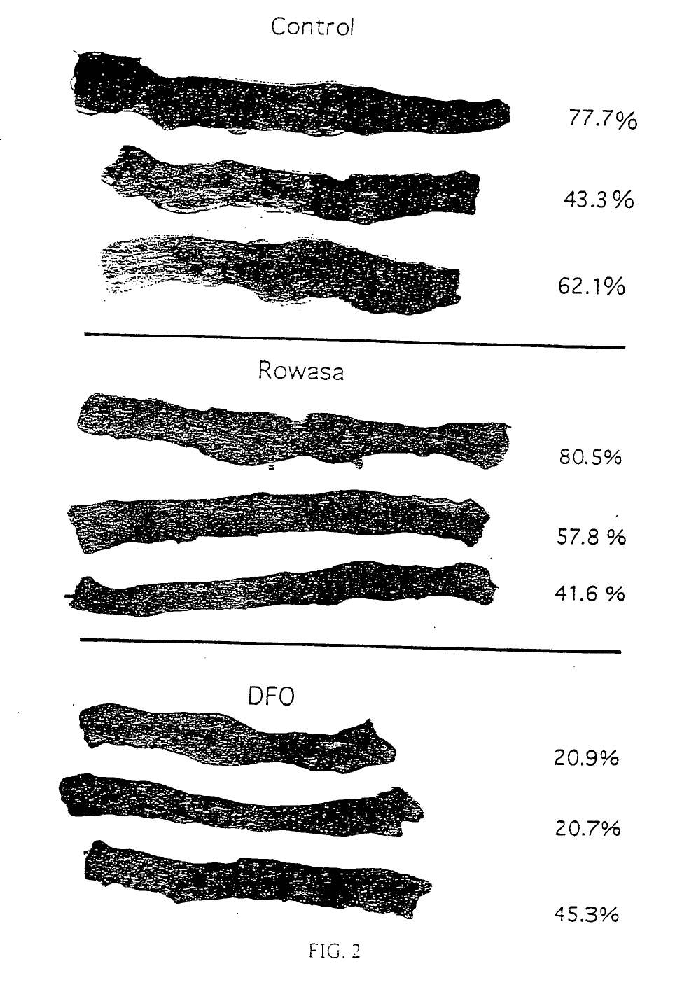

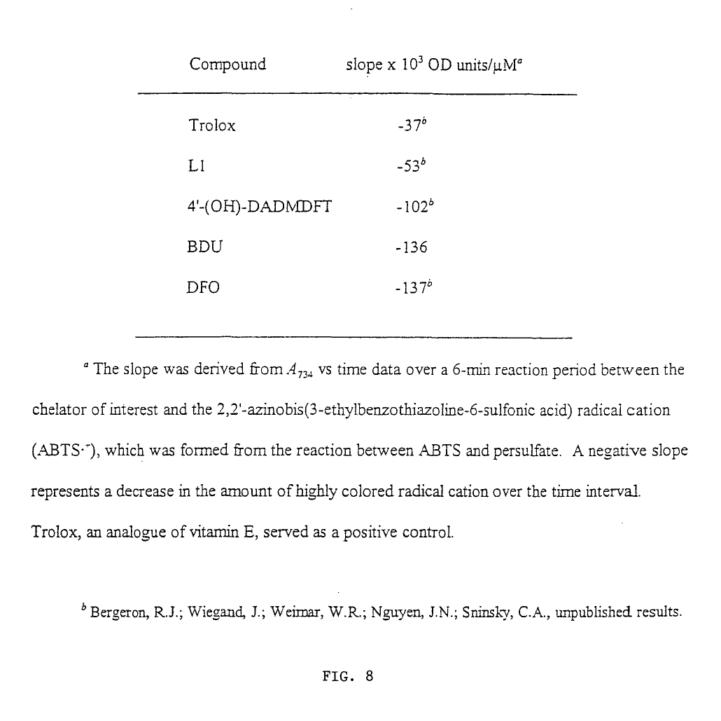

Consistent with previously reported results in a slightly different model, the colons of animals treated with DFO were similar to those of animals treated with the N-methylhydroxamate of 4′-(HO)-DADMDFT (4′-(HO)-DADMDFT—NMH); there were significant differences between the colons of DFO-treated animals and the acetic acid controls (FIGS. 2 and 8) (P<0.001 and P<0.01 by densitometry and biochemistry, respectively). In a manner similar to what was found with DMDFT, the carboxylic acid 4′-(HO)-DADFT did not protect the rats against acetic acid-induced colonic damage (P<0.05 by both measurements). Its N-methylhydroxamate (4′-(HO)-DADFT—NMH) was moderately effective (P<0.05 by both image analysis and MPO assay), although the activity of this hydroxamate was not as good as that of the other hydroxamates. Owing to this lesser degree of efficacy, the significance of the difference between 4′-(HO)-DADFT and 4′-(HO)-DADFT—NMH was equivocal, barely so as measured by densitometry (P=0.05) and not at all by the MPO assay (P>0.05). Finally, when ROWASAS, the pharmaceutical preparation which contains 5-ASA, was evaluated, it did not perform well at all (FIGS. 2 and 8). The damage observed in the colons of rats treated with this drug was remarkably similar to that in the untreated acetic acid controls.

### EXAMPLE 4

Synthesis of (S,S)-1,11-Bis[5-(4-Carboxy-4,5-Dihydrothiazol-2-Yl)-2,4-Dihydroxyphenyl]-4,8-Dioxaundecane (BDU)

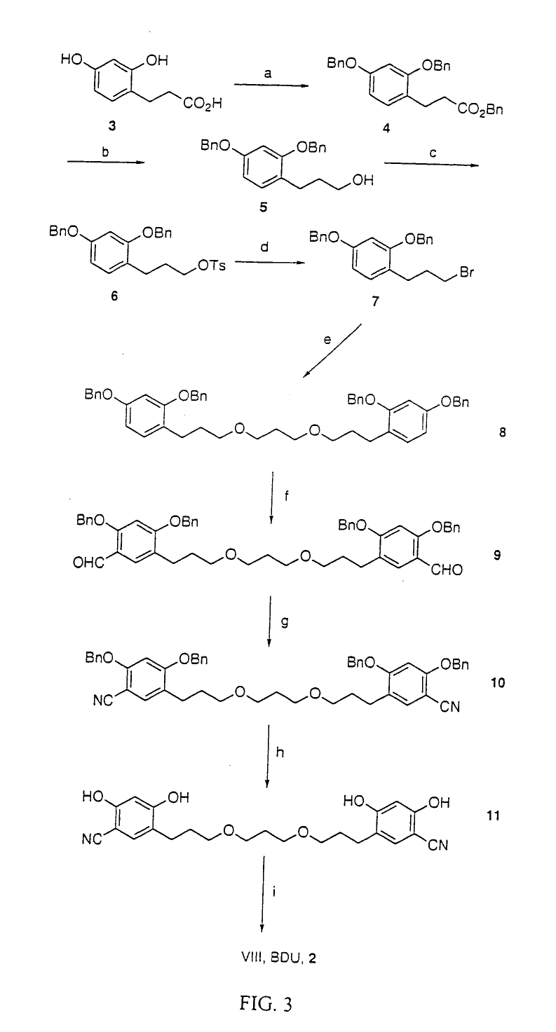

The synthetic scheme for BDU is presented in FIG. 3.

All reagents were purchased from Aldrich Chemical Co. (Milwaukee, Wis.) and were used without further purification. Fisher Optima-grade solvents were routinely used, and reactions were run under nitrogen. DMF and THF were distilled, the latter from sodium and benzophenone. Organic extracts were dried with anhydrous sodium sulfate. Silica gel 32-63 from Selecto Scientific, Inc. (Suwanee, Ga.) was used for flash column chromatography. NMR spectra were recorded at 300 MHz (¹H) or at 75 MHz (¹³C) on a Varian Unity 300. Unless otherwise indicated, the spectra were run in CDC13 with tetramethylsilane (δ 0.0 ppm) for ¹H or the solvent (δ 77.0 ppm) for ¹³C as standards. Coupling constants (J) are in hertz. Elemental analyses were performed by Atlantic Microlabs (Norcross, Ga.). Computer-based molecular modeling and energy minimizations were accomplished using SYBYL (Version 6.5, Tripos, St. Louis, Mo.) on a Silicon Graphics Indigo-2 workstation and visualized with Chem 3D (CambridgeSoft, Cambridge, Mass.) on a model 6400/200 Power Macintosh computer.

3-(2,4-Dihydroxyphenyl)propionic Acid (3). The title compound (3) was prepared by a literature method taken from Amakasu, T. and Sato, K., J. Am. Chem. Soc. 31:1433-1436 (1966). ¹H NMR (d₆-DMSO-2.49) δ 2.37 (t, 2 H, J=7.8), 2.60 (t, 2 H, J=7.8), 6.09 (dd, 1 H, J=8.4, 2.4), 6.24 (d, 1 H, J=2.4), 6.78 (d, 1 H, J=8.4), 8.96 (s, 1 H), 9.14 (br s, 1 H), 11.98 (br s, 1 H); ¹³C NMR (d₆-DMSO-39.50): δ 24.89, 34.15, 102.34, 105.84, 117.28, 129.94, 155.75, 156.53, 174.24.

Benzyl 3-(2,4-Dibenzyloxyphenyl)propionate (4). Activated K₂CO₃ (391 g, 2.83 mol) was added to a solution of 3 (128.8 g, 0.71 mol) and benzyl bromide (336 mL, 2.83 mol) in acetone (3 L), and the mixture was heated at reflux overnight. After the reaction mixture was cooled and filtered, the solid was rinsed with acetone. The filtrate was concentrated under reduced pressure; chromatography (hexanes, then 8:1 hexanes/ethyl acetate) furnished 4 (278.7 g, 87%) as a white solid: ¹H NMR δ 2.67 (t, 2 H, J=7.5), 2.96 (t, 2 H, J=7.5), 5.00 (s, 2 H), 5.03 (s, 2 H), 5.08 (s, 2 H), 6.47 (dd, 1 H, J=8.4, 2.4), 6.57 (d, 1 H, J=2.4), 7.04 (d, 1 H, J=8.4), 7.34 (m, 15 H); ¹³C NMR 6 25.66, 34.47, 66.04, 69.77, 70.15, 100.53, 105.25, 106.76, 121.67, 127.00, 127.52, 127.76, 127.96, 128.06, 128.09, 128.47, 128.53, 128.57, 130.31, 136.08, 137.00, 157.36, 158.63, 173.21; HRMS m/z calculated for C₃₀H₂₉O₄ 453.2066 (M+H), found 453.2054. Elemental analysis of C₃₀H₂₉O₄: C: calculated 79.62, found 79.70; H: calculated 6.24, found 6.31.

3-(2,4-Dibenzyloxyphenyl)propanol (5). A solution of 4 (16.64 g, 36.77 mmol) in tetrahydrofuran (THF) (150 mL) was added dropwise to LiAlH₄ (1.0 M in THF, 40.5 mL, 40.5 mmol) in THF (150 mL). After the reaction mixture was stirred overnight, H₂O (20 mL) was cautiously added. After the mixture was concentrated in vacuo, the residue was treated with 1 M HCl (150 mL) and was extracted with CH₂Cl₂ (3×150 mL). The organic extracts were washed with aqueous NaHCO₃ and brine; solvent was removed by rotary evaporation. Recrystallization from aqueous ethanol gave 5 (10.44 g, 82%) as a waxy white solid: ¹H NMR δ 1.59 (t, 1 H, J=6.3), 1.83 (m, 2 H), 2.70 (t, 2 H, J =7.5), 3.58 (q, 2 H, J=6.3), 5.02 (s, 2 H), 5.03 (s, 2 H), 6.53 (dd, 1 H, J =8.4, 2.4), 6.61 (d, 1 H, J=2.4), 7.06 (d, 1 H, J=8.4), 7.39 (m, 10 H); ¹³C NMR δ 25.42, 33.18, 61.91, 70.16, 70.19, 100.58, 105.67, 122.93, 127.30, 127.54, 127.98, 128.00, 128.58, 128.63, 130.38, 136.84, 137.02, 157.36, 158.30; HRMS m/z calculated for C₂₃H₂₅O₃ 349.1804 (M+H), found 349.1872. Elemental analysis of C₂₃H₂₄O₃: C: calculated 79.28, found 79.31; H: calculated 6.94, found 7.05.

3-(2,4-Dibenzyloxyphenyl)propylp-Tosylate (6). P-Tosyl chloride (1.14 g, 6.00 inmol in CH₂Cl₂ (20 nL) was added dropwise to 5 (1.74 g, 5.00 mmol) and pyridine (8.0 mL) in CH₂Cl₂ (40 mL), cooled in an ice-bath, and the reaction was stirred at room temperature overnight. The mixture was poured into 1 N HCl (200 mL) in an ice slurry and was extracted with CHCl₃ (200 mL). The organic layer was washed with H₂O, aqueous NaHCO₃, and brine; solvent was removed in vacuo. Purification by chromatography (CHCl₃) provided 6 (1.93 g, 77%) as a white solid: ¹H NMR δ 1.92 (m, 2 H), 2.43 (s, 3 H), 2.62 (t, 2 H, J=7.2), 4.01 (t, 2 H, J=6.3), 4.99 (s, 2 H), 5.00 (s, 2 H), 6.44 (dd, 1 H, J=8.4, 2.4), 6.56 (d, l H, J=2.4), 6.89 (d, 1 H, J =8.4), 7.37 (m, 12 H), 7.76 (m, 2 H); ¹³C NMR δ 21.60, 25.80, 28.98, 69.76, 70.15, 70.20, 100.54, 105.24, 121.63, 127.00, 127.51, 127.82, 127.86, 127.97, 128.57, 129.75, 130.36, 133.21, 136.97, 144.51, 157.29, 158.51; HRMS m/z calculated for C₃₀H₃₁O₅S 503.1892 (M+H), found 503.1885. Elemental analysis of C₃₀H₃₀O₅S: C: calculated 71.69, found 71.51; H: calculated 6.02, found 5.96.

1-(3-Bromopropyl)-2,4-dibenzyloxybenzene (7). A mixture of 6 (4.52 g, 9.00 mmol) and LiBr (3.15 g, 36.0 mmol) in acetone (300 mL) was heated at reflux overnight. The solvent was removed under reduced pressure, and the residue was taken up in diethyl ether. Treatment with H₂O and brine, solvent removal under reduced pressure, and chromatography (4:1 hexanes/ethyl acetate (EtOAc)) furnished 7 (3.29 g, 89%) as a white solid: ¹H NMR δ 2.13 (m, 2 H), 2.76 (t, 2 H, J=7.2), 3.38 (t, 2 H, J=6.6), 5.01 (s, 2 H), 5.02 (s, 2 H), 6.51 (dd, 1 H, J =8.1, 2.4), 6.59 (d, 1 H, J=2.4), 7.07 (d, 1 H, J=8.1), 7.40 (m, 10 H); ¹³C NMR δ 28.36, 32.84, 33.75, 69.83, 70.17, 100.61, 105.28, 121.83, 127.08, 127.53, 127.82, 127.96, 128.53, 128.57, 130.51, 137.00, 137.04, 157.37, 158.53; HRMS m/z calculated for C₂₃H₂₃ ⁷⁹BrO₂ 410.0882 (M), found 410.0884.

1,11-Bis(2,4-dib enzyloxyphenyl)-4,8-dioxaundecane (8). Powdered KOH (86.1%, 3.01 g, 46.2 mmol) was added to 1,3-propanediol (1.02 g, 13.4 mmol) in dimethyl sulfoxide (DMSO) (50 mL). After the mixture was stirred vigorously for 0.5 h, 7 (11.0 g, 26.8 mmol) was added. The reaction was heated at 50° C. for 0.5 hours and then was stirred at room temperature overnight. The mixture was poured into ice-cold brine (500 mL) and extracted with toluene (3×200 mL). The organic portion was washed with brine (2×500 mL) and was concentrated under reduced pressure. Chromatography (4:1 hexanes/EtOAc) afforded 8 (5.78 g, 58%) as a yellow oil: ¹H NMR δ 1.84 (m, 6 H), 2.67 (t, 4 H, J=7.5), 3.41 (t, 4 H, J=6.6), 3.46 (t, 4 H, J=6.3), 4.99 (s, 4 H), 5.01 (s, 4 H), 6.49 (dd, 2 H, J=8.1, 2.4), 6.58 (d, 2 H, J =2.4), 7.04 (d, 2 H, J =8.1), 7.39 (m, 20 H); ¹³C NMR δ 26.31, 29.85, 30.20, 67.71, 69.75, 70.13, 70.42, 100.51, 105.16, 123.35, 127.00, 127.54, 127.69, 127.91, 128.48, 128.54, 130.18, 137.09, 137.23, 157.35, 158.18; HRMS m/z calculated for C₄₉H₅₃O₆ 737.3842 (M+H), found 737.3819.

1,11-Bis (2,4-dibenzyloxy-5-formylphenyl)-4,8-dioxaundecane (9). Phosphorus oxychloride (5.808 g, 37.88 mmol) in CH₃CN (80 mL) was added dropwise to DMF (3.251 g, 44.47 mmol) and CH₃CN (16 mL), and the mixture was stirred at room temperature for 1 hour. Compound 8 (12.14 g, 16.47 mmol) in CH₃CN (80 mL) was slowly added. The reaction was stirred at room temperature for 1 hour, refluxed overnight, and concentrated under reduced pressure. The residue was treated with H₂O (100 mL) and 1,4-dioxane (100 mL), heated at 50° C. for 2 hours, and concentrated in vacuo. The residue was dissolved in ethyl acetate (500 mL), washed with brine (500 mL), and concentrated by rotary evaporation. Chromatography (2:1 hexanes/EtOAc) gave 9 (7.96 g, 61%) as a white solid: ¹H NMR δ 1.82 (m, 6 H), 2.65 (t, 4 H, J=7.2), 3.40 (t, 4 H, J =6.3), 3.44 (t, 4 H, J=6.3), 5.09 (s, 4 H), 5.10 (s, 4 H), 6.49 (s, 2 H), 7.38 (m, 20 H), 7.65 (s, 2 H), 10.36 (s, 2 H); ¹³C NMR δ 26.16, 29.46, 30.18, 67.75, 70.18, 70.32, 70.79, 97.20, 118.56, 124.08, 126.98, 127.21, 128.14, 128.24, 128.71, 129.48, 136.14, 161.59, 162.78, 188.23; HRMS m/z calculated for C₅₁H₅₃O₈ 793.3740 (M+H), found 793.3815.

1,11-Bis(5-cyano-2,4-dibenzyloxyphenyl)-4,8-dioxaundecane (10). A solution of 9 (20.42 g, 25.8 mmol), hydroxylamine hydrochloride (3.95 g, 56.8 mmol), and triethylamine (6.26 g, 61.9 mmol) in CH₃CN (500 mL) was stirred at 45° C. overnight. Phthalic anhydride (11.5 g, 77.4 mmol) was added, and the mixture was heated at reflux overnight. After the solution was concentrated under reduced pressure, the residue was diluted with CH₂Cl₂ (600 mL) and washed with aqueous NaHCO₃ (600 mL) and brine (600 mL). Solvent removal and chromatography (3:1 hexanes/EtOAc) afforded 10 (15.64 g, 77%) as a white solid: ¹H NMR δ 1.80 (m, 6 H), 2.62 (t, 4 H, J=7.5), 3.38 (t, 4 H, J =6.3), 3.45 (t, 4 H, J=6.3), 5.03 (s, 4 H), 5.12 (s, 4 H), 6.47 (s, 2 H), 7.28 (s, 2 H), 7.36 (m, 20 H); ¹³C NMR δ 26.04, 29.24, 30.12, 67.71, 70.01, 70.14, 70.87, 93.46, 97.96, 117.09, 124.18, 126.95, 128.17, 128.20, 128.71, 133.98, 135.80, 135.88, 160.58, 160.93; HRMS m/z calculated for C₅₁H₅₁N₂O₆ 787.3747 (M +H), found 787.3745.

1,11-Bis(5-cyano-2,4-dihydroxyphenyl)-4,8-dioxaundecane (11). Palladium on activated carbon (10%, 3.14 g) was added to a solution of 10 (5.23 g, 6.65 mmol) in ethyl acetate (500 mL) and iron-free ethanol (100 mL), and the suspension was stirred under H₂ (1 atm) at room temperature for 5.5 hours. The reaction mixture was heated on a steam bath and was filtered through Celite. The filtrate was concentrated in vacuo; chromatography (20:3 CHCl₃/CH₃OH) gave 11 (2.55 g, 90%) as a white solid: ¹H NMR (d₆-DMSO-2.49) δ 1.69 (m, 6 H), 2.42 (t, 4 H, J=7.5), 3.30 (t, 4 H, J=6.6), 3.38 (t, 4 H, J=6.6), 6.47 (s, 2 H), 7.17 (s, 2 H), 10.30 (s, 2 H), 10.54 (s, 2 H); ¹³C NMR (d₆-DMSO-39.50); δ 25.34, 28.99, 29.71, 67.06, 69.50, 88.82, 102.11, 117.97, 120.72, 133.40, 159.94, 160.60; HRMS m/z calculated for C₂₃H₂₇N₂O₆ 427.1869 (M+H), found 427.1845.

(S,S)-1,11-Bis[5-(4-carboxy-4,5-dihydrothiazol-2-yl)-2,4-dihydroxyphenyl]-4,8-dioxaundecane (2). Distilled solvents and glassware that had been presoaked in 3 N HCl for 15 min. were employed. D—Cysteine hydrochloride monohydrate (1.23 g, 7.02 mmol) was added to 11 (1.00 g, 2.34 mmol) in degassed CH₃OH (20 mL) and 0.1 M phosphate buffer at pH 6.0 (15 mL). Sodium bicarbonate (0.590 g, 7.02 mmol) was carefully added, and the mixture was stirred at reflux for 2 days. The reaction mixture was concentrated under reduced pressure, H₂O was added, and the pH was adjusted to 2 by addition of 10% citric acid solution. Solid was filtered and recrystallized from aqueous ethanol to furnish 2 (0.81 g, 55%) as a beige powder: ¹H NMR (d₆-DMSO-2.49) δ 1.72 (m, 6 H), 2.48 (t, 4 H, J=7.2), 3.32 (t, 4 H, J =6.3), 3.41 (t, 4 H, J =6.3) 3.54 (dd, 1 H, J=7.2, 11.1), 3.61 (dd, 1 H, J=9.3, 11.1) 5.34 (dd, 2 H, J=7.2, 9.3), 6.36 (s, 2 H), 7.03 (s, 2 H), 10.24 (br s, 2 H), 12.45 (br s, 2 H), 13.04 (br s, 2 H); ¹³C NMR (d₆-DMSO-39.50) δ 25.50, 29.19, 29.78, 33.15, 67.17, 69.25, 75.91, 102.00, 107.64, 120.11, 131.49, 158.55, 160.17, 171.57, 171.95; HRMS m/z calculated for C₂₉H₃₅N₂O₁₀S₂ 635.1733 (M+H), found 635.1696. Elemental analysis of C₂₉H₃₄N₂O₁₀S₂: C: calculated 54.88, found 54.17; H: calculated 5.40, found 5.45; N: calculated 4.41, found 4.40. Optical rotation: α²⁴_{D}+3.1 (c 1.06, DMF).

### EXAMPLE 5

Prevention of Iron-Mediated Oxidation of Ascorbate

Nitrilotriacetic acid (NTA), 1,2-dimethyl-3-hydroxypyridin-4-one (L1), desferrioxamine B (DFO), (S)-4′-(HO)-DADMDFT, and (S,S)-1,11-Bis[5-(4-carboxy-4,5-dihydrothiazol-2-yl)-2,4-dihydroxyphenyl]-4,8-dioxaundecane (BDU) were tested for their ability to diminish the iron-mediated oxidation of ascorbate by the method of Dean and Nicholson, Free Radical Res. 20: 83-101 (1994). Briefly, a solution of freshly prepared ascorbate (100 μM) in sodium phosphate buffer (5 mM, pH 7.4) was incubated in the presence of FeCl₃ (30 μM) and a compound of interest (compound/Fe ratios varied from 0-3) for 40 min. The A₂₆₅ was read at 10 and 40 min; the ΔA265 in the presence of compound was compared to that in its absence.

The measurement examines the disappearance of ascorbate. It is known that DFO prevents ascorbate-mediated reduction of Fe(III); it serves as a positive control in the present study. Both NTA and L1 promote ascorbate-mediated reduction of Fe(III) and serve as negative controls.

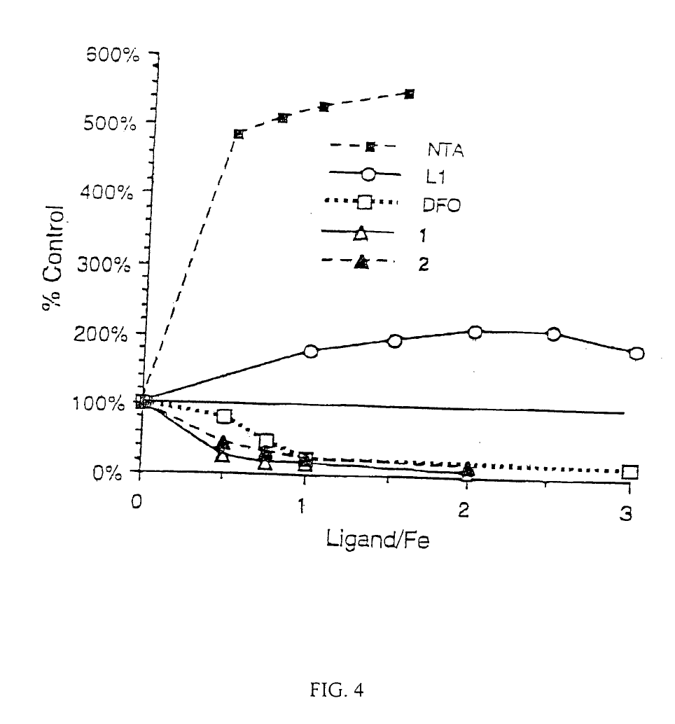

Consistent with others' findings, NTA exerted a profoundly stimulatory effect on reduction of Fe(III); L1 also promoted the reaction, although not as dramatically, at compound:metal ratios of up to 3: 1. Iron(III) reduction was inhibited by DFO at compound:metal ratios of less than 1: 1, although the optimum effect was seen at 1:1. Whereas both desferrithiocin analogues provided significant protection at compound:metal ratios of less than 1, (S)-4′-(HO)-DADMDFT was significantly (P<0.005) less inhibitory than BDU (FIG. 4).

### EXAMPLE 6

Quenching of the ABTS Radical Cation.

Compounds were tested for their ability to quench the radical cation formed from 2,2′-azinobis(3-ethylbenzothiazoline-6-sulfonic acid) (ABTS) by a published method (Re, R., et al., Free Radical Biol. Med. 26:1231-1237 (1999). Briefly, a stock solution of ABTS radical cation was generated by mixing ABTS (10 mM, 2.10 mL) with K₂S₂O₈ (8.17 mM, 0.90 mL) in H₂O and allowing the solution to sit in the dark at room temperature for 18 hours. This stock solution of deep blue-green ABTS radical cation was diluted in sufficient sodium phosphate (10 mM, pH 7.4) to give an A₇₃₄ of about 0.900. Test compounds were added to a final concentration ranging from 1.25 to 15 μM, and the decrease in A₇₃₄ was read after 1, 2, 4 and 6 min. The reaction was largely complete by 1 min, but the data presented are based on a 6-min. reaction time.

This radical cation decolorization assay utilizes the pre-formed radical monocation of 2,2′-azinobis(3-ethylbenzothiazoline-6-sulfonic acid) (ABTS) and has been used to evaluate the antioxidant capacity of a large number of compounds and mixtures. Briefly, the change in absorbance of the blue-green chromophore was recorded after the addition of the compound of interest at each of the different concentrations, and the slope of the ΔA734 vs. compound concentration line was calculated. The positive control for this reaction was Trolox, an analogue of vitamin E. The decrease in A₇₃₄ as a function of compound concentration is the comparitor among the five compounds evaluated. Trolox, L1, DFO, (S)-4′-(HO)-DADMDFT and BDU (FIG. 8). All four of the compounds performed better than Trolox; DFO BDU>(S)-4′-(HO)-DADMDFT>L1>Trolox. Thus, all of these compounds could be expected to serve as excellent radical scavengers.

### EXAMPLE 7

Prevention of Iron-Mediated Oxidation of Ascorbate

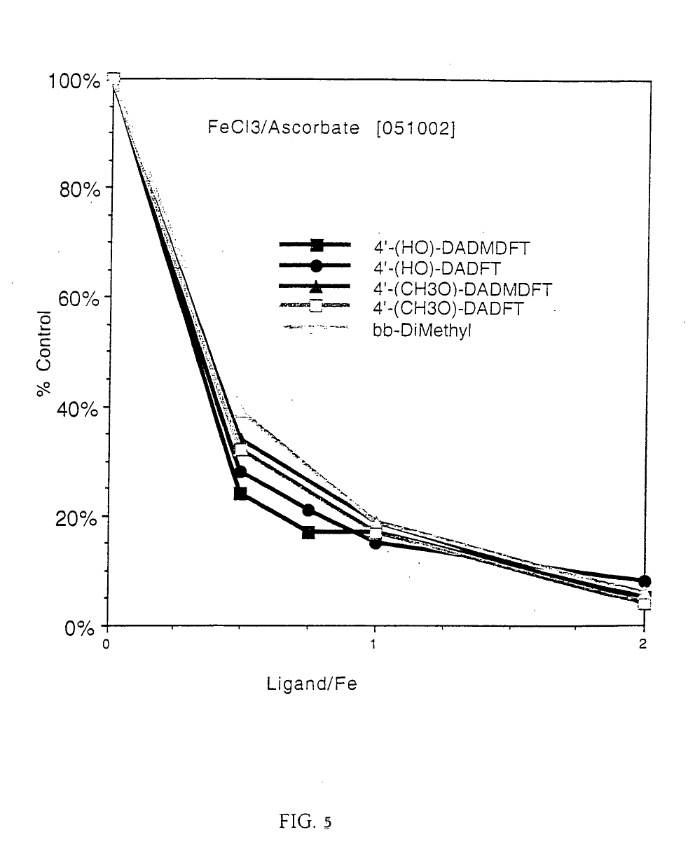

4′-methoxydesazadesmethyldesferrithiocin (4′-(CH₃0)-DADMDFT) and 4-methoxydesazadesferrithiocin (4′-(CH₃O)-DADFT) were tested as described in Example 5. Both of these analogues slowed Fe(III) reduction considerably (FIG. 5).

### EXAMPLE 8

Quenching of the ABTS Radical Cation

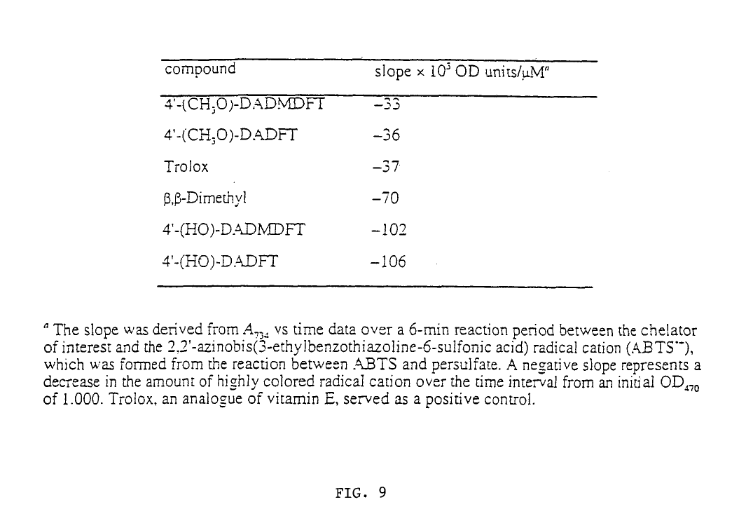

4′-(CH₃O)-DADMDFT and 4′-(CH₃O)-DADFT were evaluated by the method described in Example 6. The 4′-methoxylated compounds were less effective radical scavengers than the corresponding 4′-hydroxylated molecules. Nevertheless, both 4′-(CH₃O)-DADMDFT and 4′-(CH₃O)-DADFT were as effective as Trolox at trapping free radicals (FIG. 9).

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of suppressing radical formation in vitro, comprising the step of contacting a solution with a compound represented by Structural Formula (I): 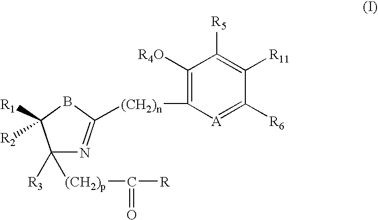
wherein:
R is —OH, —OR₇, —N(OH)R₈;
R₁ is —H, —CH₃, or an alkyl of 1-6 carbons;
R₂ is —H, —CH₃, or an alkyl of 1-6 carbons;
R₃ is —H or —CH₃, or an alkyl of 1-6 carbons, or R₂ and R₃ together form a double bond;
R₄ is —H, acyl of 1-4 carbons, or alkyl of 1-4 carbons;
R₅ is —H, —OH, —O-acyl of 1-4 carbons, —O-alkyl of 1-4 carbons, or —L—X;
R₆ is —H, —OH, alkyl of 1-6 carbons, a halogen, —L—Y, or R₆ is —C═C—C═C—, which together with R₁₁ forms a fused ring system as follows: 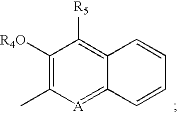
R₇ is alkyl of one to four carbons or optionally substituted benzyl;
R₈ is —H, alkyl of one to four carbons, optionally substituted benzyl, 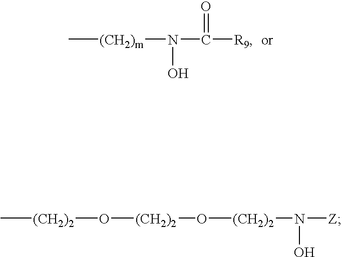
R₉ is —H, alkyl of one to four carbons, or optionally substituted benzyl;
R₁₁ is —H, —OH, —O-acyl of 1-4 carbons, or —O-alkyl of 1-4 carbons;
A is N, CH, or C(OH);
B is S, O, NR₉, CH₂ or CH₂S;
L is an alkylene group of 3 to about 20 carbon atoms which is optionally interrupted by one or more oxygen atoms;
a is 2 or 3;
m is an integer from 1 to 8;
n is 0, 1 or 2;
p is 0, 1 or 2;
X is 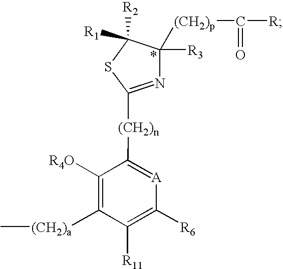
Y is 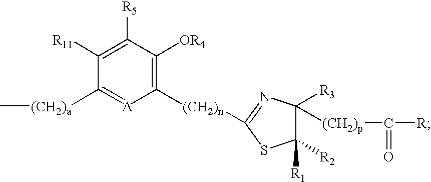
and
Z is 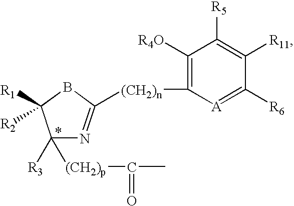
wherein each of the substituents shown is defined above;
or a compound of formula (I) wherein the ring containing the B and N moieties is fully reduced and contains no double bonds; or a pharmaceutically acceptable salt of the compound represented by formula (I) or a stereoisomer of the compound or mixture of stereoisomers; with the proviso that when R is —OH, R₁ and R₂ are —H, R₃ is —CH₃, R₄, R₅, R₆, and R₁₁ are —H, A is N, and B is S, then n and p are not 0.

2. A method of suppressing radical formation in vitro, comprising the step of contacting a solution with a compound represented by Structural Formula (II) or Structural Formula (III): 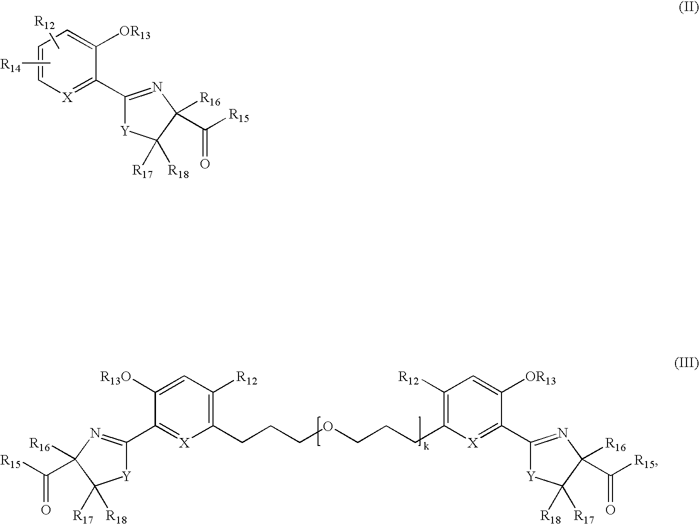
wherein:
R₁₂ is —H, —OR₁₉, or an —O-acyl group;
R₁₃ is —H, —R₁₉, or an acyl group;
R₁₄, R₁₆, R₁₇, R₁₈, R₁₉, and R₂₀ are each independently —H or a lower substituted or unsubstituted alkyl group, or R₁₆ and R₁₈ together form a double bond;
R₁₅ is —OH, —OR₂₀, or —N(R₂₀)OH;
X is CH or N;
Y is S, CH, O, NR₂₀, or SCH₂; and
k is an integer;
or a pharmaceutically acceptable salt thereof, provided that for compounds represented by Structural Formula (II) when R₁₂, R₁₃ and R₁₄ are —H, R₁₅ is —OH, R₁₆ is —CH₃, R₁₇ and R₁₈ are —H, and X is N, then Y is not S.

3. The method of claim 2, wherein wherein iron(III) is present and the ratio of the compound to iron is greater than or equal to about 0.25.

4. The method of claim 3, wherein the ratio of the compound to iron is greater than or equal to about 0.5 and less than or equal to about 2.0.

5. The method of claim 2, wherein hydrogen peroxide, an organic peroxide, or a nitrosothiol is present.

6. The method of claim 2, wherein R₁₂ is —H, —OH, or —OCH₃; R₁₃ is —H; R₁₄, R_{16,} R_{17,} R₁₈, and R₂₀ are each independently —H or —CH₃; R₁₅ is —OH or —N(R₂₀)OH; and k is 1 or 2.

7. The method of claim 6, wherein the compound is represented by a structural formula selected from the group consisting of:

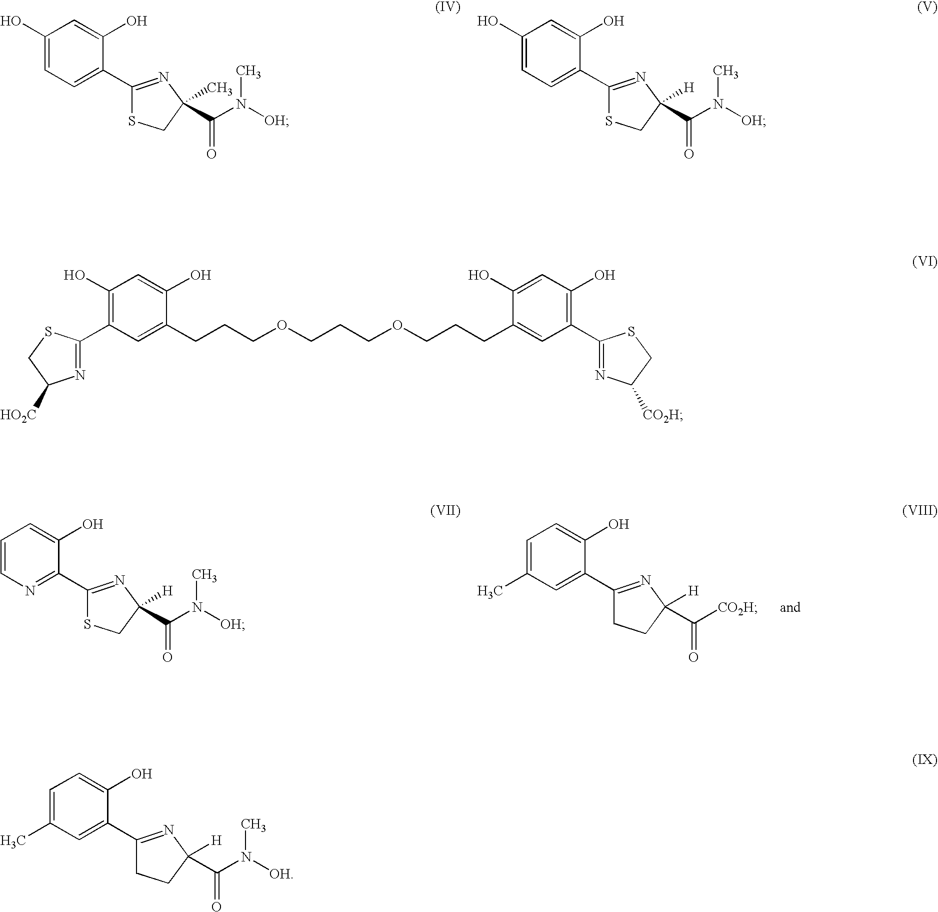

8. The method of claim 7, wherein the compound is represented by a structural formula selected from the group consisting of:

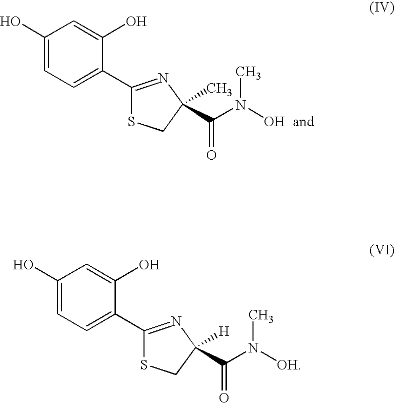

9. A method of treating a patient to suppress radical formation, provided said patient is not suffering from inflammatory bowel disorder or trivalent metal overload, comprising the step of administering to said patient a therapeutically effective amount of a compound represented by Structural Formula (I): 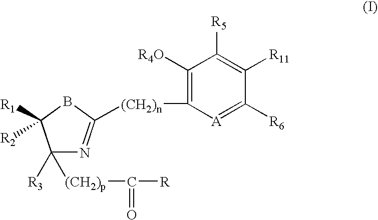
wherein:
R is —OH, —OR₇, —N(OH)R₈;
R₁ is —H, —CH₃, or an alkyl of 1-6 carbons;
R₂ is —H, —CH₃, or an alkyl of 1-6 carbons;
R₃ is —H, —CH₃, or an alkyl of 1-6 carbons, or R₂ and R₃ together form a double bond;
R₄ is —H, acyl of 1-4 carbons, or alkyl of 1-4 carbons;
R₅ is —H, —OH, —O-acyl of 1-4 carbons, —O-alkyl of 1-4 carbons, or —L—X;
R₆ is —H, —OH, alkyl of 1-6 carbons, a halogen, —L—Y, or R₆ is —C═C—C═C—, which together with R₁₁ forms a fused ring system as follows 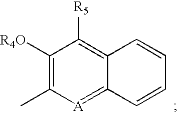
R₇ is alkyl of one to four carbons or optionally substituted benzyl;
R₈ is —H, alkyl of one to four carbons, optionally substituted benzyl, 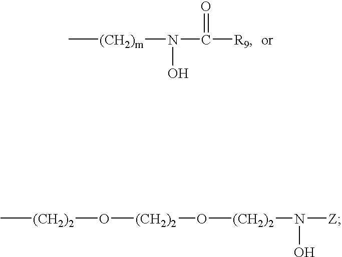
R₉ is —H, alkyl of one to four carbons, or optionally substituted benzyl;
R₁₁ is —H, —OH, —O-acyl of 1-4 carbons, or —O-alkyl of 1-4 carbons;
A is N, CH, or C(OH);
B is S, O, NR₉, CH₂ or CH₂S;
L is an alkylene group of 3 to about 20 carbon atoms which is optionally interrupted by one or more oxygen atoms;
a is 2 or 3;
m is an integer from 1 to 8;
n is 0, 1 or 2;
p is 0, 1 or 2;
X is 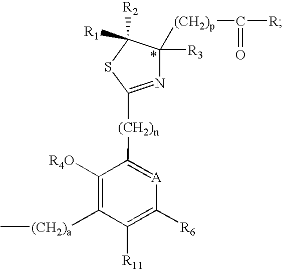
Y is 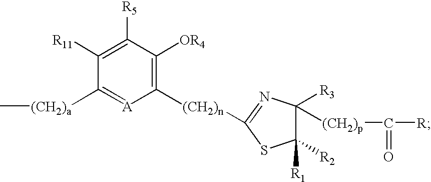
and
Z is 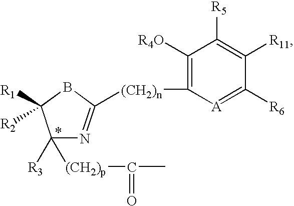
wherein each of the substituents shown is defined above;
or a compound of formula (I) wherein the ring containing the B and N moieties is fully reduced and contains no double bonds; or a pharmaceutically acceptable salt of the compound represented by formula (I) or a stereoisomer of the compound or mixture of stereoisomers; with the proviso that when R is —OH, R₁ and R₂ are —H, R₃ is —CH₃, R₄, R₅, R₆, and R₁₁ are —H, A is N, and B is S, then n and p are not 0.

10. A method of treating a patient to suppress radical formation, provided that said patient is not suffering from inflammatory bowel disorder or trivalent metal overload, comprising the step of administering to said patient a therapeutically effective amount of a compound represented by Structural Formula (II) or Structural Formula (III): 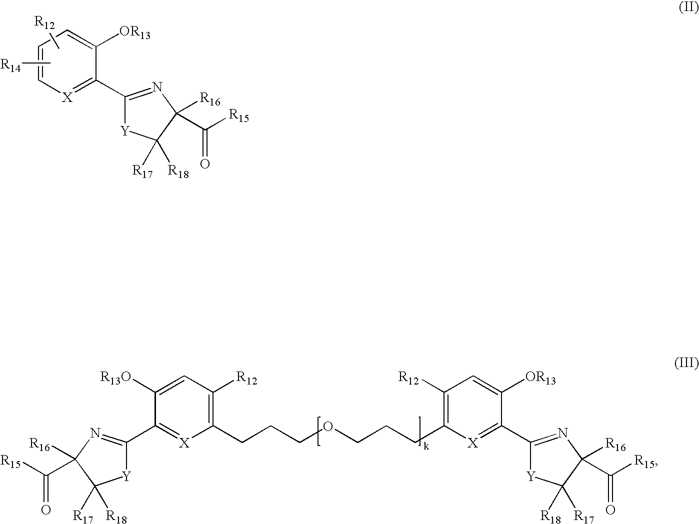
wherein:
R₁₂ is —H, —OR₁₉, or an —O-acyl group;
R₁₃ is —H, —R₁₉, or an acyl group;
R₁₄, R₁₆, R₁₇, R₁₈, R₁₉, and R₂₀ are each independently —H or a lower substituted or unsubstituted alkyl group, or R₁₆ and R₁₈ together form a double bond;
R₁₅ is —OH, —OR₂₀, or —N(R₂₀)OH;
X is CH or N;
Y is S, CH, O, NR₂₀, or SCH₂; and
k is an integer;
or a pharmaceutically acceptable salt thereof, provided that for compounds represented by Structural Formula (II) when R₁₂, R₁₃ and R₁₄ are —H, R₁₅ is —OH, R₁₆ is —CH₃, R₁₇ and R₁₈ are —H, and X is N, then Y is not S.

11. A method of treating a patient who is suffering from, has suffered from, or is at risk of suffering from an ischemic episode, comprising the step of administering to said patient a therapeutically effective amount of a compound represented by Structural Formula (I): 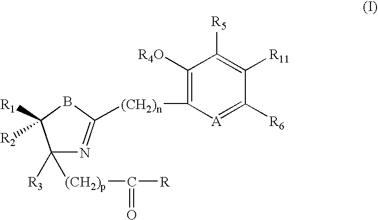
wherein:
R is —OH, —OR₇, —N(OH)R₈;
R₁ is —H, —CH₃, or an alkyl of 1-6 carbons;
R₂ is —H, —CH₃, or an alkyl of 1-6 carbons;
R₃ is —H, —CH₃, or an alkyl of 1-6 carbons, or R₂ and R₃ together form a double bond;
R₄ is —H, acyl of 1-4 carbons, or alkyl of 1-4 carbons;
R₅ is —H, —OH, —O-acyl of 1-4 carbons, —O-alkyl of 1-4 carbons, or —L—X;
R₆ is —H, —OH, alkyl of 1-6 carbons, a halogen, —L—Y, or R₆ is —C═C—C═C—, which together with R₁₁ forms a fused ring system as follows: 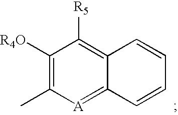
R₇ is alkyl of one to four carbons or optionally substituted benzyl;
R₈ is —H, alkyl of one to four carbons, optionally substituted benzyl, 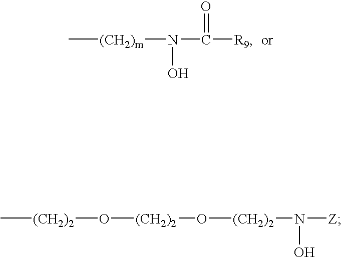
R₉ is —H, alkyl of one to four carbons, or optionally substituted benzyl;
R₁₁ is —H, —OH, —O-acyl of 1-4 carbons, or —O-alkyl of 1-4 carbons;
A is N, CH, or C(OH);
B is S, O, NR₉, CH₂ or CH₂S;
L is an alkylene group of 3 to about 20 carbon atoms which is optionally interrupted by one or more oxygen atoms;
a is 2 or 3;
m is an integer from 1 to 8;
n is 0, 1 or 2;
p is 0, 1 or 2;
X is 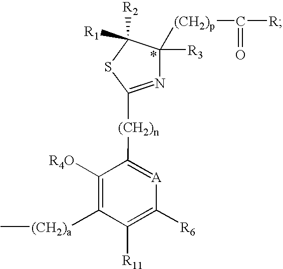
Y is 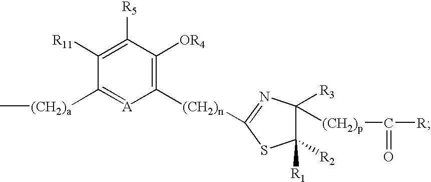
and
Z is 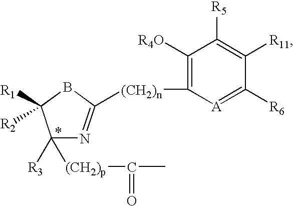
wherein each of the substituents shown is defined above;
or a compound of formula (I) wherein the ring containing the B and N moieties is fully reduced and contains no double bonds; or a pharmaceutically acceptable salt of the compound represented by formula (I) or a stereoisomer of the compound or mixture of stereoisomers.

12. A method of treating a patient who is suffering from, has suffered from, or is at risk of suffering from an ischemic episode, comprising the step of administering to said patient a therapeutically effective amount of a compound represented by Structural Formula (II) or Structural Formula (III): 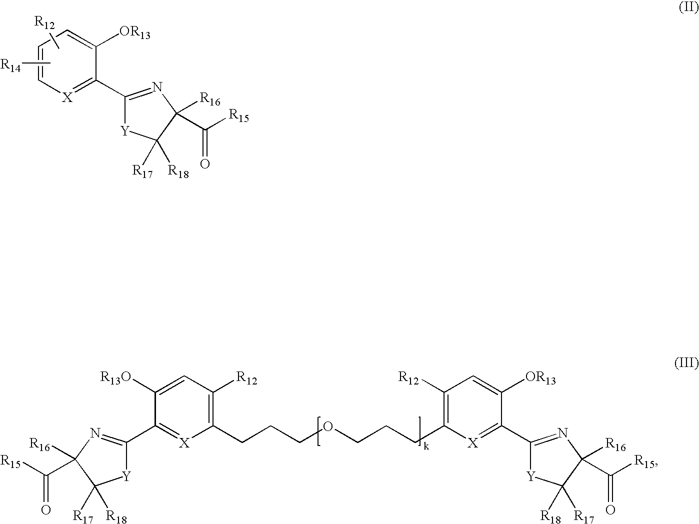
wherein:
R₁₂ is —H, —OR₁₉, or an —O-acyl group;
R₁₃ is —H, —R₁₉, or an acyl group;
R₁₄, R₁₆, R₁₇, R₁₈, R₁₉, and R₂₀ are each independently —H or a lower substituted or unsubstituted alkyl group, or R₁₆ and R₁₈ together form a double bond;
R₁₅ is —OH, —OR₂₀, or —N(R₂₀)OH;
X is CH or N;
Y is S, CH, O, NR₂₀, or SCH₂; and
k is an integer;
or a pharmaceutically acceptable salt thereof, provided that for compounds represented by Structural Formula (II) when R₁₂, R₁₃ and R₁₄ are —H, R₁₅ is —OH, R₁₆ is —CH₃, R₁₇ and R₁₈ are —H, and X is N, then Y is not S.

13. A method of treating a patient who is suffering from an inflammatory disorder, provided said inflammatory disorder is not inflammatory bowel disorder, comprising the step of administering to said patient a therapeutically effective amount of a compound represented by Structural Formula (I): 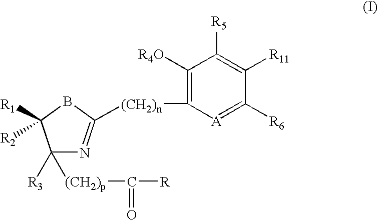
wherein:
R is —OH, —OR₇, —N(OH)R₈;
R₁ is —H, —CH₃, or an alkyl of 1-6 carbons;
R₂ is —H, —CH₃, or an alkyl of 1-6 carbons;
R₃ is —H, —CH₃, or an alkyl of 1-6 carbons, or R₂ and R₃ together form a double bond;
R₄ is —H, acyl of 1-4 carbons, or alkyl of 1-4 carbons;
R₅ is —H, —OH, —O-acyl of 1-4 carbons, —O-alkyl of 1-4 carbons, or —L—X;
R₆ is —H, —OH, alkyl of 1-6 carbons, a halogen, —L—Y, or R₆ is —C═C—C═C—, which together with R₁ forms a fused ring system as follows: 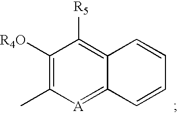
R₇ is alkyl of one to four carbons or optionally substituted benzyl;
R₈ is —H, alkyl of one to four carbons, optionally substituted benzyl, 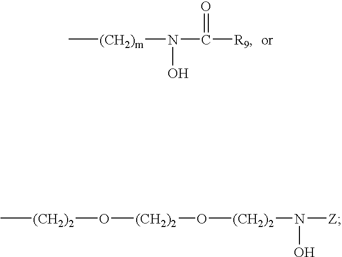
R₉ is —H, alkyl of one to four carbons, or optionally substituted benzyl;
R₁₁ is —H, —OH, —O-acyl of 1-4 carbons, or —O-alkyl of 1-4 carbons;
A is N, CH, or C(OH);
B is S, O, NR_{9,} CH₂or CH₂S;
L is an alkylene group of 3 to about 20 carbon atoms which is optionally interrupted by one or more oxygen atoms;
a is 2 or 3;
m is an integer from 1 to 8;
n is 0, 1 or 2;
p is 0, 1 or 2;
X is 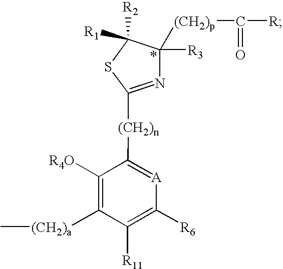
Y is 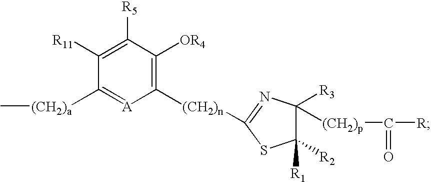
and
Z is 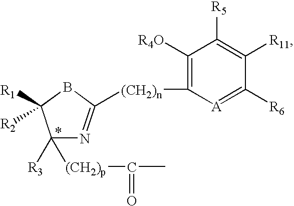
wherein each of the substituents shown is defined above;
or a compound of formula (I) wherein the ring containing the B and N moieties is fully reduced and contains no double bonds; or a pharmaceutically acceptable salt of the compound represented by formula (I) or a stereoisomer of the compound or mixture of stereoisomers; with the proviso that when R is —OH, R₁ and R₂ are —H, R₃ is —CH₃, R₄, R₅, R₆, and R₁₁ are —H, A is N, and B is S, then n and p are not 0.

14. A method of treating a patient who is suffering from an inflammatory disorder, provided said inflammatory disorder is not inflammatory bowel disorder, comprising the step of administering to said patient a therapeutically effective amount of a compound represented by Structural Formula (II) or Structural Formula (III): 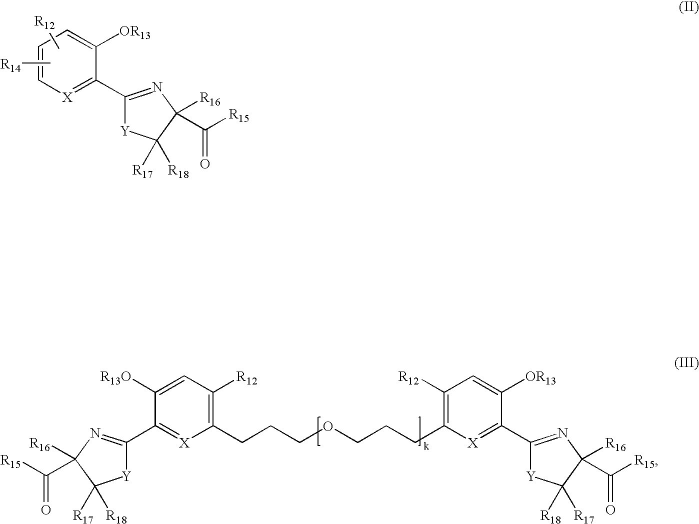
wherein:
R₁₂ is —H, —OR₁₉, or an —O-acyl group;
R₁₃ is —H, —R₁₉, or an acyl group;
R₁₄, R₁₆, R₁₇, R₁₈, R₁₉, and R₂₀ are each independently —H or a lower substituted or unsubstituted alkyl group, or R₁₆ and R₁₈ together form a double bond;
R₁₅ is —OH, —OR₂₀, or —N(R₂₀)OH;
X is CH or N;
Y is S, CH, O, NR₂₀, or SCH₂; and
k is an integer;
or a pharmaceutically acceptable salt thereof, provided that for compounds represented by Structural Formula (II) when R₁₂, R₁₃ and R₁₄ are —H, R₁₅ is —OH, R₁₆ is —CH₃, R₁₇ and R₁₈ are —H, and X is N, then Y is not S.

15. A method of treating a patient who is suffering from neoplastic disease or a preneoplastic condition, comprising the step of administering to said patient a therapeutically effective amount of a compound represented by Structural Formula (I): 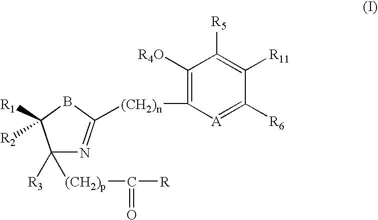
wherein:
R is —OH, —OR₇, —N(OH)R₈;
R₁ is —H, —CH₃, or an alkyl of 1-6 carbons;
R₂ is —H, —CH₃, or an alkyl of 1-6 carbons;
R₃ is —H, —CH₃, or an alkyl of 1-6 carbons, or R₂ and R₃ together form a double bond;
R₄ is —H, acyl of 1-4 carbons, or alkyl of 1-4 carbons;
R₅ is —H, —OH, —O-acyl of 1-4 carbons, —O-alkyl of 1-4 carbons, or —L—X;
R₆ is —H, —OH, alkyl of 1-6 carbons, a halogen, —L—Y, or R₆ is —C═C—C═C—, which together with R₁₁ forms a fused ring system as follows: 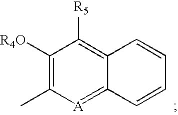
R₇ is alkyl of one to four carbons or optionally substituted benzyl;
R₈ is —H, alkyl of one to four carbons, optionally substituted benzyl, 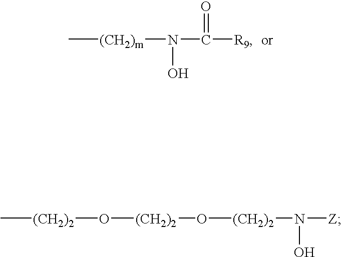
R₉ is —H, alkyl of one to four carbons, or optionally substituted benzyl;
R₁₁ is —H, —OH, —O-acyl of 1-4 carbons, or —O-alkyl of 1-4 carbons;
A is N, CH, or C(OH);
B is S, O, NR₉, CH₂ or CH₂S;
L is an alkylene group of 3 to about 20 carbon atoms which is optionally interrupted by one or more oxygen atoms;
a is 2 or 3;
m is an integer from 1 to 8;
n is 0, 1 or 2;
p is 0, 1 or 2;
X is 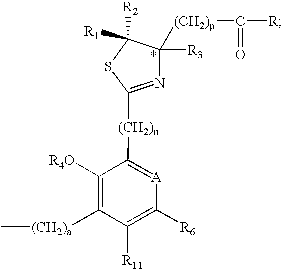
Y is 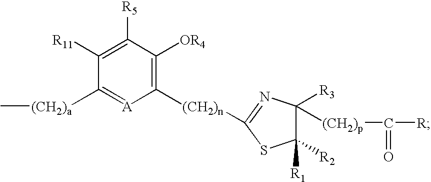
and
Z is 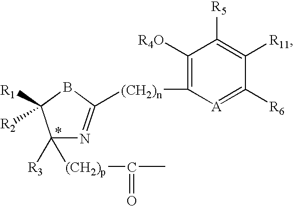
wherein each of the substituents shown is defined above;
or a compound of formula (I) wherein the ring containing the B and N moieties is fully reduced and contains no double bonds; or a pharmaceutically acceptable salt of the compound represented by formula (I) or a stereoisomer of the compound or mixture of stereoisomers; with the proviso that when R is —OH, R₁ and R₂ are —H, R₃ is —H or —CH₃, R₄, R₅, R₆, and R₁₁ are —H, A is N, and B is S, then n and p are not 0, and when R is —OH, R₁, R₂ and R₃ are —H, R₄, R₅, R₆, and R₁₁ are —H, A is CH, and B is S, then n and p are not 0.

16. A method of treating a patient who is suffering from neoplastic disease or a preneoplastic condition, comprising the step of administering to said patient a therapeutically effective amount of a compound represented by represented by Structural Formula (II) or Structural Formula (III): 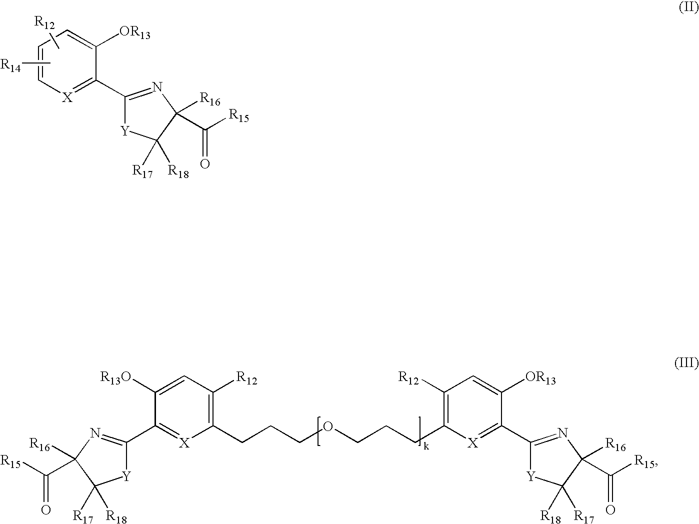
wherein:
R₁₂ is —H, —OR₁₉, or an —O-acyl group;
R₃ is —H, —R₁₉, or an acyl group;
R₁₄, R₁₆, R₁₇, R₁₈, R₁₉, and R₂₀ are each independently —H or a lower substituted or unsubstituted alkyl group, or R₁₆ and R₁₈ together form a double bond;
R₁₅ is —OH, —OR₂₀, or —N(R₂₀)OH;
X is CH or N;
Y is S, CH, O, NR₂₀, or SCH₂; and
k is an integer;
or a pharmaceutically acceptable salt thereof, provided that for compounds represented by Structural Formula (II) when R₁₂ and R₁₄ are —H, R₁₃ is —H or —CH₃, R₁₅ is —OH, R₁₆ is —CH₃, R₁₇ and R₁₈ are —H, and X is N, then Y is not S, and when R₁₂, R₁₃ and R₁₄ are —H, R₁₅ is —OH, R₁₆ is —CH₃, R₁₇ and R₁₈ are —H, and X is CH, then Y is not S.

17. A method of preventing or inhibiting oxidation of a substance in vitro, comprising the step of contacting said substance with an effective amount of an antioxidant represented by Structural Formula (I): 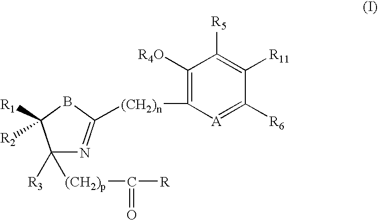
wherein:
R is —OH, —OR₇, —N(OH)R₈;
R₁ is —H, —CH₃, or an alkyl of 1-6 carbons;
R₂ is —H, —CH₃, or an alkyl of 1-6 carbons;
R₃ is —H, —CH₃, or an alkyl of 1-6 carbons, or R₂ and R₃ together form a double bond;
R₄ is —H, acyl of 1-4 carbons, or alkyl of 1-4 carbons;
R₅ is —H, —OH, —O-acyl of 1-4 carbons, —O-alkyl of 1-4 carbons, or —L—X;
R₆ is —H, —OH, alkyl of 1-6 carbons, a halogen, —L—Y, or R₆ is —C═C—C═C—, which together with R₁ forms a fused ring system as follows: 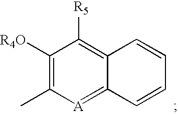
R₇ is alkyl of one to four carbons or optionally substituted benzyl;
R₈ is —H, alkyl of one to four carbons, optionally substituted benzyl, 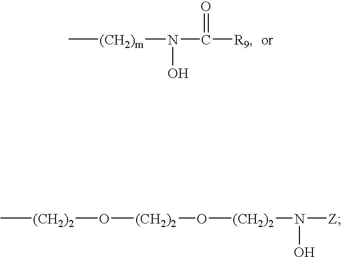
R₉ is —H, alkyl of one to four carbons, or optionally substituted benzyl;
R₁₁ is —H, —OH, —O-acyl of 1-4 carbons, or —O-alkyl of 1-4 carbons;
A is N, CH, or C(OH);
B is S, O, NR₉, CH₂ or CH₂S;
L is an alkylene group of 3 to about 20 carbon atoms which is optionally interrupted by one or more oxygen atoms;
a is 2 or 3;
m is an integer from 1 to 8;
n is 0, 1 or 2;
p is 0, 1 or 2;
X is 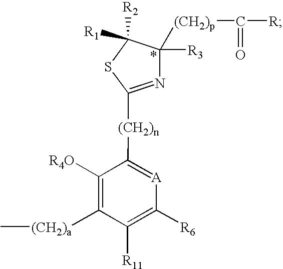
Y is 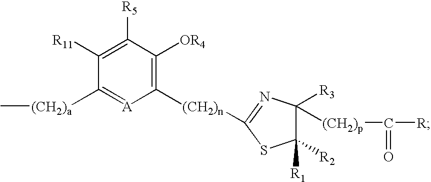
and
Z is 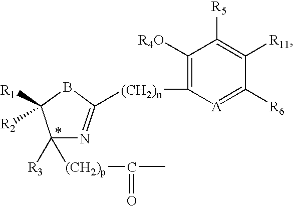
wherein each of the substituents shown is defined above;
or a compound of formula (I) wherein the ring containing the B and N moieties is fully reduced and contains no double bonds; or a pharmaceutically acceptable salt of the compound represented by formula (I) or a stereoisomer of the compound or mixture of stereoisomers; with the proviso that when R is —OH, R₁ and R₂ are —H, R₃ is —CH₃, R₄, R₅, R₆, and R₁₁ are —H, A is N, and B is S, then n and p are not 0.

18. A method of preventing or inhibiting oxidation of a substance in vitro, comprising the step of contacting said substance with an effective amount of a antioxidant represented by Structural Formula (II) or Structural Formula (III): 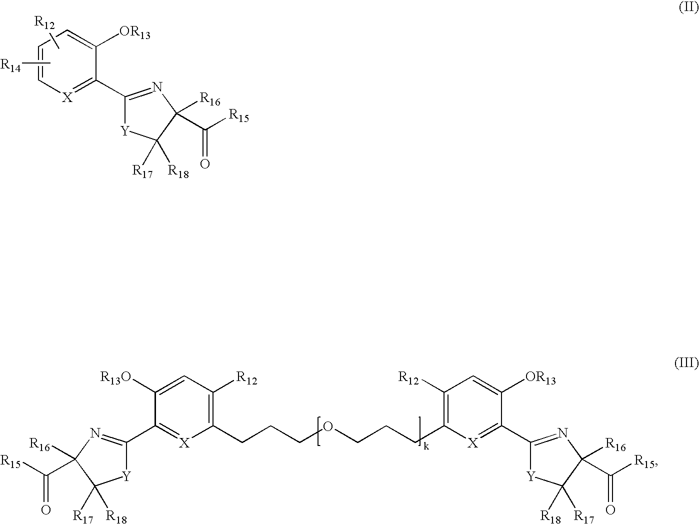
wherein:
R₁₂ is —H, —OR₁₉, or an —O-acyl group;
R₁₃ is —H, —R₁₉, or an acyl group;
R₁₄, R₁₆, R₁₇, R₁₈, R₁₉, and R₂₀ are each independently —H or a lower substituted or unsubstituted alkyl group, or R₁₆ and R₁₈ together form a double bond;
R₁₅ is —OH, —OR₂₀, or —N(R₂₀)OH;
X is CH or N;
Y is S, CH, O, NR₂₀, or SCH₂; and
k is an integer;
or a pharmaceutically acceptable salt thereof, provided that for compounds represented by Structural Formula (II) when R₁₂, R₁₃ and R₁₄ are —H, R₁₅ is —OH, R₁₆ is —CH₃, R₁₇ and R₁₈ are —H, and X is N, then Y is not S.

19. The method of claim 18, wherein R₁₂ is —H, —OH, —OCH₃; R₁₃ is —H; R₁₄, R₁₆, R₁₇, R₁₈, and R₂₀ are each independently —H or —CH₃; R₁₅ is —OH or —N(R₂₀)OH; and k is 1 or2.

20. The method of claim 19, wherein the substance is a food product.

21. The method of claim 19, wherein the antioxidant is represented by a structural formula selected from the group consisting of:

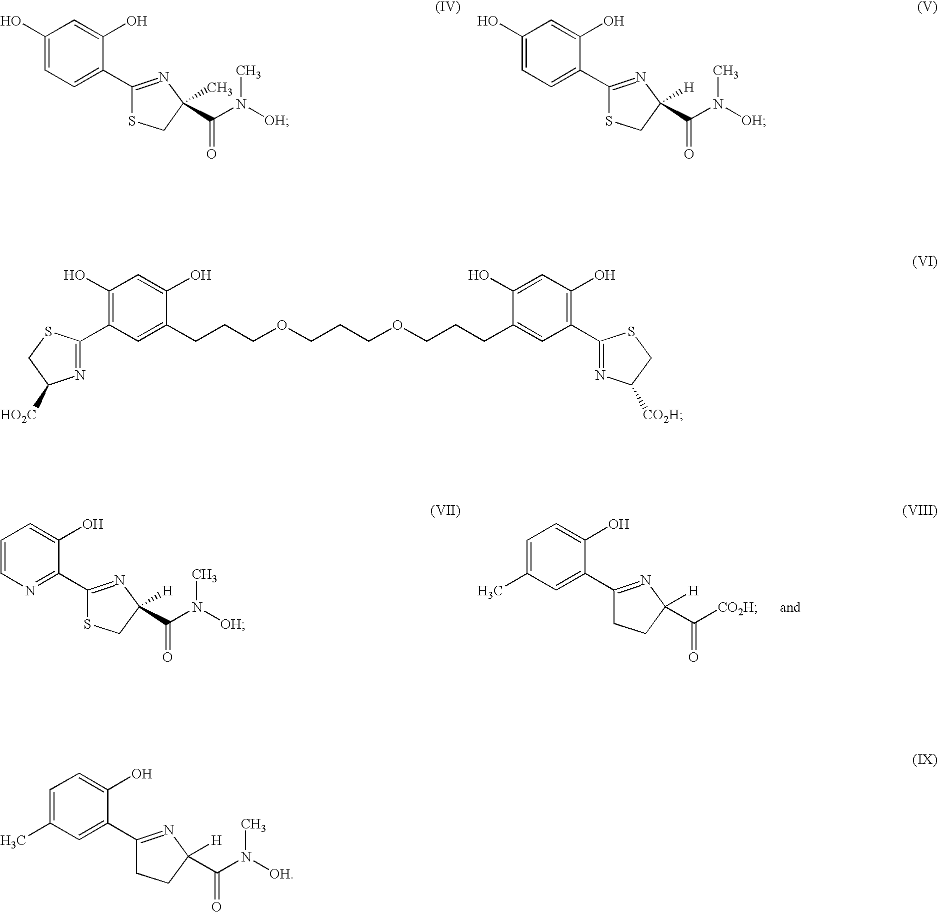

22. The method of claim 21, wherein the antioxidant is represented by the structural formula:

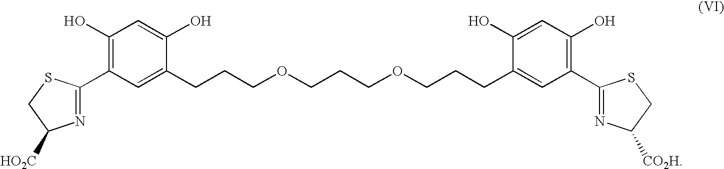

23. A method of treating a patient in need of antioxidant therapy with a compound represented by Structural Formula (I), provided said patient does not suffer from inflammatory bowel disorder or trivalent metal overload, comprising the step of administering to the patient a therapeutically effective amount of a compound represented by Structural Formula (I): 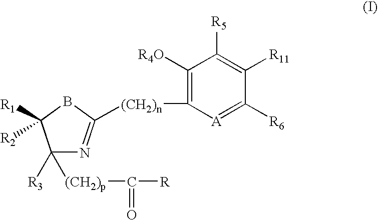
wherein:
R is —OH, —OR₇, —N(OH)R₈;
R₁ is —H, —CH₃, or an alkyl of 1-6 carbons;
R₂ is —H, —CH₃, or an alkyl of 1-6 carbons;
R₃ is —H, —CH₃, or an alkyl of 1-6 carbons, or R₂ and R₃ together form a double bond;
R₄ is —H, acyl of 1-4 carbons, or alkyl of 1-4 carbons;
R₅ is —H, —OH, —O-acyl of 1-4 carbons, —O-alkyl of 1-4 carbons, or —L—X;
R₆ is —H, —OH, alkyl of 1-6 carbons, a halogen, —L—Y, or R₆ is —C═C—C═C—, which together with R₁₁ forms a fused ring system as follows: 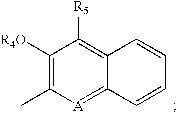
R₇ is alkyl of one to four carbons or optionally substituted benzyl;
R₈ is —H, alkyl of one to four carbons, optionally substituted benzyl, 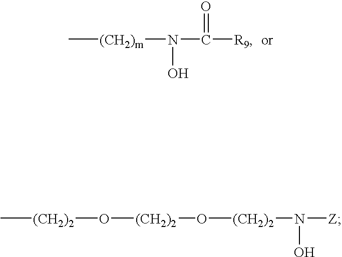
R₉ is —H, alkyl of one to four carbons, or optionally substituted benzyl;
R₁₁ is —H, —OH, —O-acyl of 1-4 carbons, or —O-alkyl of 1-4 carbons;
A is N, CH, or C(OH);
B is S, O, NR₉, CH₂ or CH₂S;
L is an alkylene group of 3 to about 20 carbon atoms which is optionally interrupted by one or more oxygen atoms;
a is 2 or 3;
m is an integer from 1 to 8;
n is 0, 1 or 2;
p is 0, 1 or 2;
X is 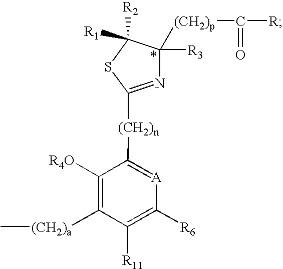
Y is 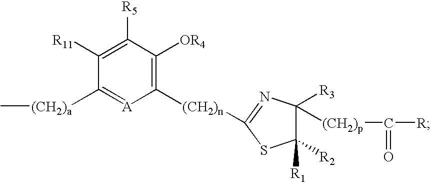
and
Z is 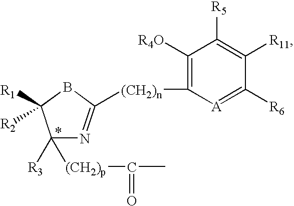
wherein each of the substituents shown is defined above;
or a compound of formula (I) wherein the ring containing the B and N moieties is fully reduced and contains no double bonds; or a pharmaceutically acceptable salt of the compound represented by formula (I) or a stereoisomer of the compound or mixture of stereoisomers; with the proviso that when R is —OH, R₁ and R₂ are —H, R₃ is —CH₃, R₄, R₅, R₆, and R₁₁ are —H, A is N, and B is S, then n and p are not 0.

24. A method of treating a patient in need of antioxidant therapy with a compound represented by Structural Formula (II) or Structural Formula (III), provided said patient does not suffer from inflammatory bowel disorder or trivalent metal overload, comprising the step of administering the patient a therapeutically effective amount of a compound represented by Structural Formula (II) or (III): 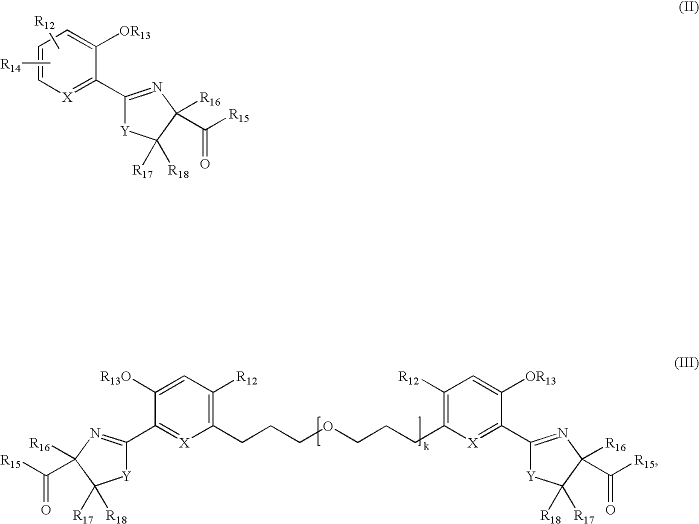
wherein:
R₁₂ is —H, —OR₁₉, or an —O-acyl group;
R₁₃ is —H, —R₁₉, or an acyl group;
R₁₄, R₁₆, R₁₇, R₁₈, R₁₉, and R₂₀ are each independently —H or a lower substituted or unsubstituted alkyl group, or R₁₆ and R₁₈ together form a double bond;
R₁₅ is —OH, —OR₂₀, or —N(R₂₀)OH;
X is CH or N;
Y is S, CH, O, NR₂₀, or SCH₂; and
k is an integer;
or a pharmaceutically acceptable salt thereof, provided that for compounds represented by Structural Formula (II) when R_{12,} R₁₃ and R₁₄ are —H, R₁₅ is —OH, R₁₆ is —CH₃, R₁₇ and R₁₈ are —H, and X is N, then Y is not S.

25. The method of claim 24, wherein R₁₂ is —H, —OH, or —OCH₃; R₁₃ is —H; R₁₄, R₁₆, R₁₇, R₁₈, and R₂₀ are each independently —H or —CH₃; R₁₅ is —OH or —N(R₂₀)OH; and k is 1 or 2.

26. The method of claim 25, wherein the patient in need of antioxidant therapy has or is at risk of having elevated levels of reactive oxygen species.

27. The method of claim 26, wherein the reactive oxygen species are selected from the group consisting of superoxide, hydrogen peroxide, an organic peroxide, hydroxyl radical, hydrogen peroxyl radical, an organic peroxyl radical, singlet oxygen, or a combination thereof.

28. A method of scavenging free radicals, comprising the step of contacting said free radicals with a compound represented by Structural Formula (I): 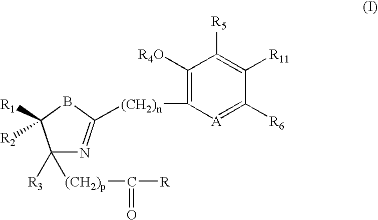
wherein:
R is —OH, —OR₇, —N(OH)R₈;
R₁ is —H, —CH₃, or an alkyl of 1-6 carbons;
R₂ is —H, —CH₃, or an alkyl of 1-6 carbons;
R₃ is —H, —CH₃, or an alkyl of 1-6 carbons, or R₂ and R₃ together form a double bond;
R₄ is —H, acyl of 1-4 carbons, or alkyl of 1-4 carbons;
R₅ is —H, —OH, —O-acyl of 1-4 carbons, —O-alkyl of 1-4 carbons, or-L-X;
R₆ is —H, —OH, alkyl of 1-6 carbons, a halogen, —L—Y, or R₆ is —C═C—C═C—, which together with R₁₁ forms a fused ring system as follows: 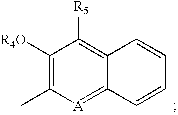
R₇ is alkyl of one to four carbons or optionally substituted benzyl;
R₈ is —H, alkyl of one to four carbons, optionally substituted benzyl, 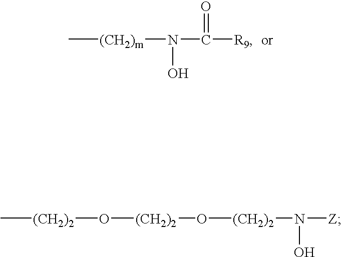
R₉ is —H, alkyl of one to four carbons, or optionally substituted benzyl;
R₁₁ is —H, —OH, —O-acyl of 1-4 carbons, or —O-alkyl of 1-4 carbons;
A is N, CH, or C(OH);
B is S, O, NR₉, CH₂ or CH₂S;
L is an alkylene group of 3 to about 20 carbon atoms which is optionally interrupted by one or more oxygen atoms;
a is 2 or 3;
m is an integer from 1 to 8;
n is 0, 1 or 2;
p is 0, 1 or 2;
X is 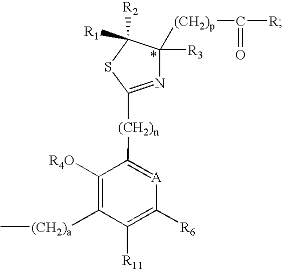
Y is 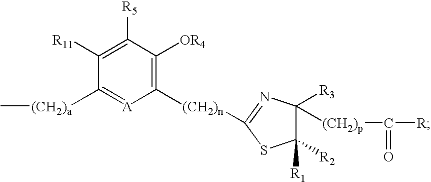
and
Z is 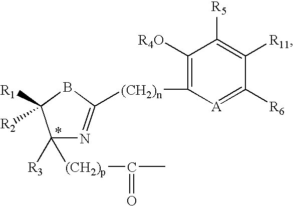
wherein each of the substituents shown is defined above;
or a compound of formula (I) wherein the ring containing the B and N moieties is fully reduced and contains no double bonds; or a pharmaceutically acceptable salt of the compound represented by formula (I) or a stereoisomer of the compound or mixture of stereoisomers; with the proviso that when R is —OH, R₁ and R₂ are —H, R₃ is —CH₃, R₄, R₅, R₆, and R₁₁ are —H, A is N, and B is S, then n and p are not 0.

29. A method of scavenging free radicals, comprising the step of contacting said free radicals with a compound represented by Structural Formula (II) or Structural Formula (III): 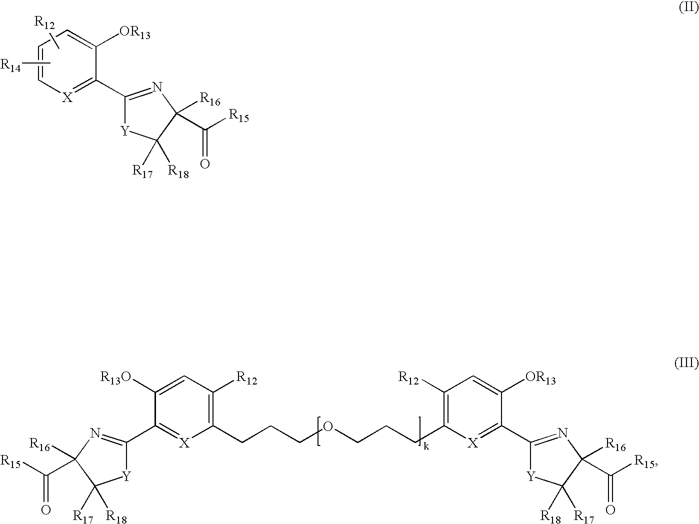
wherein:
R₁₂ is —H, —OR₁₉, or an —O-acyl group;
R₁₃ is —H, —R₁₉, or an acyl group;
R₁₄, R₁₆, R₁₇, R₁₈, R₁₉, and R₂₀ are each independently —H or a lower substituted or unsubstituted alkyl group, or R₁₆ and R₁₈ together form a double bond;
R₁₅ is —OH, —OR₂₀, or —N(R₂₀)OH;
X is CH or N;
Y is S, CH, O, NR₂₀, or SCH₂; and
k is an integer;
or a pharmaceutically acceptable salt thereof, provided that for compounds represented by Structural Formula (II) when R₁₂, R₁₃ and R₁₄ are —H, R₁₅ is —OH, R₁₆ is —CH₃, R₁₇ and R₁₈ are —H, and X is N, then Y is not S.

30. The method of claim 29, wherein said scavenging prevents or inhibits free radical-mediated damage to cells, tissues or organs.

31. The method of claim 30, wherein the free radicals are selected from the group consisting of hydroxyl radical, hydrogen peroxyl radical, organic radical, organic hydroxyl radical, organic peroxyl radical, and combinations thereof.

32. The method of claim 1, wherein R₈ is —H, alkyl of one to four carbons, optionally substituted benzyl, or

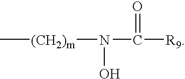

33. The method of claim 9, wherein R₈ is —H, alkyl of one to four carbons, optionally substituted benzyl, or

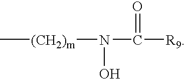

34. The method of claim 15, wherein R₈ is —H, alkyl of one to four carbons, optionally substituted benzyl, or

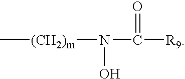

35. The method of claim 17, wherein R₈ is —H, alkyl of one to four carbons, optionally substituted benzyl, or

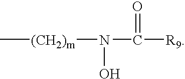

36. The method of claim 23, wherein R₈ is —H, alkyl of one to four carbons, optionally substituted benzyl, or

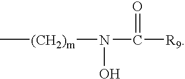

37. The method of claim 28, wherein R₈ is —H, alkyl of one to four carbons, optionally substituted benzyl, or [chemical structure]
